(19) United States
(12) Patent Application Publication
(10) Pub. No.: US 2013/0190190 A1
(43) Pub. Date: Jul. 25, 2013

(54) MOLECULAR TARGETS FOR MODULATING INTRAOCCULAR PRESSURE AND DIFFERENTIATION OF STEROID RESPONSES VERSUS NON-RESPONDERS

(71) Applicants: Stephen G. Schwartz, Naples, FL (US); Elizabeth M. Fini, Miami, FL (US)

(72) Inventors: Stephen G. Schwartz, Naples, FL (US); Elizabeth M. Fini, Miami, FL (US)

(21) Appl. No.: 12/745,666

(22) PCT Filed: Dec. 4, 2008
(86) PCT No.: PCT/US2008/085489

§ 371 Date: Sep. 24, 2010

Related U.S. Application Data
(60) Provisional application No. 60/992,204, filed on Dec. 4, 2007.

(51) Int. Cl. C12Q 1/68

(52) U.S. Cl. CPC C12Q 1/6881
USPC 506/2; 536/23.5; 435/6.11; 530/389.8; 506/17; 506/18; 536/24.5; 506/9; 536/23.2

(57) ABSTRACT

Molecular biomarkers as indicators of responses to steroids, drug targets, predictors of steroid responses and identification of drugs for modulating intraocular pressure. Kits and methods of identifying and distinguishing between steroid responders and non-responders.

### CROSS-REFERENCE TO RELATED APPLICATIONS

The present application claims the priority of U.S. provisional patent application No. 60/992,204 entitled filed Dec. 4, 2007, which is incorporated herein by reference in its entirety.

### FIELD OF THE INVENTION

Embodiments of this invention relates to molecular biomarkers, drug targets, prediction of steroid responses and modulation of intraocular pressure.

### BACKGROUND

Glaucoma is a set of diseases characterized by cupping of the optic nerve head, loss of axons from the optic nerve, and the death of retinal ganglion cells that project these axons. This characteristic set of features is called glaucomatous optic neuropathy. Elevated intraocular pressure in the eye is the major risk factor for glaucoma.

Corticosteroid treatment has become a common treatment for various diseases of the eye, including macular diseases, uveitis, and dry eye disease. The drug is delivered directly to the eye by a variety of means including drops, intravitreal injection, and slow release from a surgically-implanted device. This greatly minimizes the systemic side-effects of corticosteroid treatment. A major complication of corticosteroid treatment is an increase in intraocular pressure (TOP). This increase can be quite dramatic, and can rapidly lead to optic neuropathy if not managed.

### SUMMARY

This Summary is provided to present a summary of the invention to briefly indicate the nature and substance of the invention. It is submitted with the understanding that it will not be used to interpret or limit the scope or meaning of the claims.

Embodiments of the invention provides for a composition of molecular biomarkers identified by single nucleotide polymorphisms. These markers are shown to have a pharmacogenomic relationship between steroid-induced IOP elevation and a set of single nucleotide polymorphisms that can be used to predict steroid response. Each of these SNPs is associated with steroid response at a statistical significance between about 10⁻² and 10⁻⁸.

These SNPs mostly lie in non-coding regions of specific genes. The human genome is composed of haplotype blocks in which set of SNPs stay together during evolution, non-causative SNPs may remain with causative polymorphisms and can thus serve as phenotype predictors. Moreover, they also identify genes that are potentially involved in control of intraocular pressure. These genes, once characterized more fully, can serve as drug targets for controlling intraocular pressure and glaucoma.

Other aspects of the invention are described infra.

### BRIEF DESCRIPTION OF THE DRAWINGS

FIG. 1 is a schematic illustration showing the steps one of the methods used in identifying biomarkers.

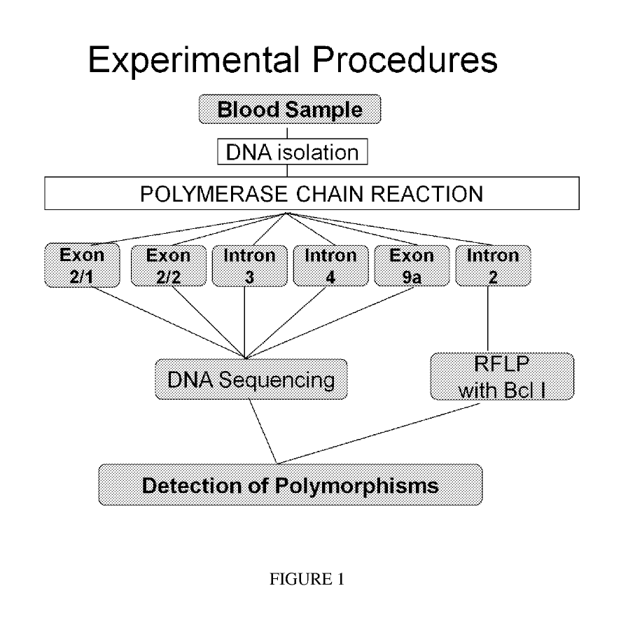

FIG. 2 is a photograph of a gel showing some of the results obtained.

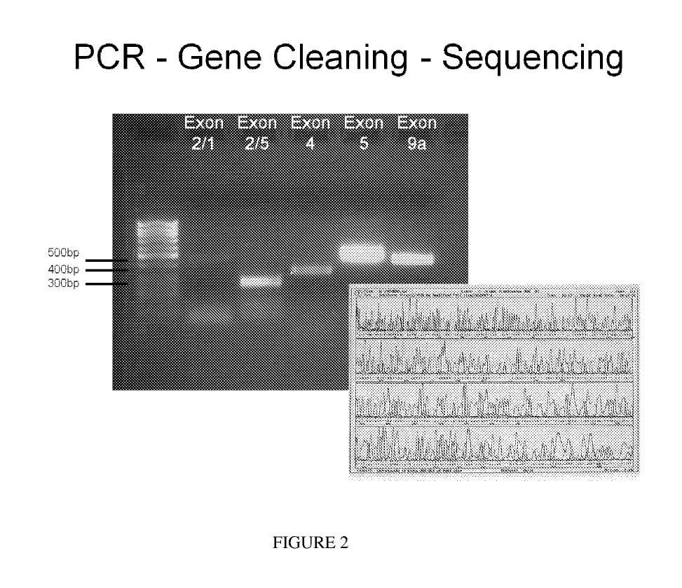

FIG. 3 is a schematic representation showing a steroid response risk e.g. intraocular pressure.

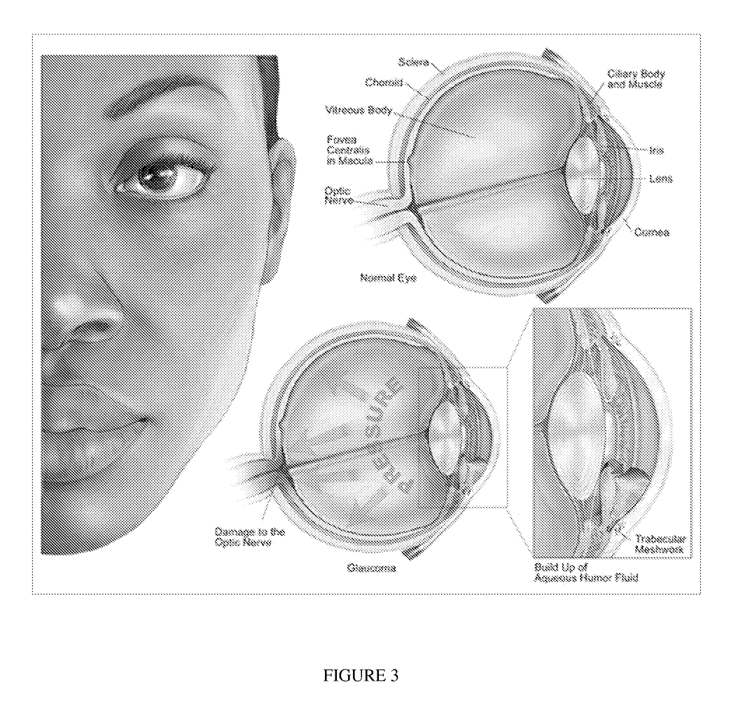

### DETAILED DESCRIPTION

The invention comprises a molecular signature of biomarkers comprising single nucleotide polymorphisms (SNP) in the identification of patients that would not respond to steroids. A major complication of corticosteroid treatment is an increase in intraocular pressure (TOP). This increase can be quite dramatic, and can rapidly lead to optic neuropathy if not managed. A medically significant corticosteroid response occurs in about 40% of patients, however, it has not been possible, up to now, to predict which patients will respond to steroids. Methods and kits for the identification of responders and non-responders prior to treatment, are provided.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the term “test substance” or “candidate therapeutic agent” are used interchangeably herein, and the terms are meant to encompass any molecule, chemical entity, composition, drug, therapeutic agent, chemotherapeutic agent, or biological agent capable of preventing, ameliorating, or treating a disease or other medical condition. The term includes small molecule compounds, antisense reagents, siRNA reagents, antibodies, and the like. A test substance can be assayed in accordance with the methods of the invention at any stage during clinical trials, during pre-trial testing, or following FDA-approval.

As used herein, the term “gene” means the gene and all currently known variants thereof and any further variants which may be elucidated, including different species.

A “biomarker polynucleotide” (or nucleic acid) as used herein, refers to a molecule comprising a nucleotide sequence, for example, as disclosed in Tables 1-3. These markers comprise at least one polymorphism, preferably, single nucleotide polymorphisms (SNPs), however, embodiments of the invention are not limited to just SNPs. Specifically included are DNA and RNA molecules obtained from cellular, cell-free, or synthetic sources, as well as genomic and cDNA sequences, unspliced or partly spliced transcripts, and splicing products. Also included are “protein nucleic acids” (PNAs) formed by conjugating bases to an amino acid backbone. The nucleic acid sequences of the invention may be single- or double-stranded (i.e., DNA-DNA, DNA-RNA and RNA-RNA hybrids), and may represent the sense or antisense strand (i.e., complementary sequences). Nucleic acids (e.g., fragments, alleles, homologs, variants, and derivatives thereof) encoding functional equivalents of a biomarker polypeptide are also embraced by the present invention.

A “biomarker polypeptide” (or protein) refers to a molecule comprising an amino acid sequence of a nucleic acid sequence, for example, polynucleotides in Tables 1-3, which may be obtained from any species, preferably mammalian, and more preferably, human, and from a variety of sources, including cellular, cell-free, synthetic, semi-synthetic, or recombinant sources. Functional equivalents of a polypeptide (e.g., fragments, variants, and derivatives thereof) are also embraced by the present invention.

As used herein, a “molecular signature” or “signature” or “biomarker profile” refers to the nucleic acids, mutants, variants, fragments and peptides thereof identified in Tables 1 to 3. However, this list can be expanded, or amended as more nucleic acid molecules are indentified.

“Variant” polynucleotides and polypeptides include molecules containing one or more deletions, insertions and/or substitutions compared to the nucleic acids in, for example, Tables 1-3. Variant polynucleotides can encode the same or a functionally-equivalent biomarker polypeptide. The term “variant” when used in context of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

“Derivative” polynucleotides include nucleic acids subjected to chemical modification, for example, replacement of hydrogen by an alkyl, acyl, or amino group. Derivatives, e.g., derivative oligonucleotides, may comprise non-naturally-occurring portions, such as altered sugar moieties or inter-sugar linkages. Exemplary among these are phosphorothioate and other sulfur containing species which are known in the art. Derivative nucleic acids may also contain labels, including radionucleotides, enzymes, fluorescent agents, chemiluminescent agents, chromogenic agents, substrates, cofactors, inhibitors, magnetic particles, and the like.

A “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

“Oligonucleotides” or “oligomers” refer to a nucleic acids, preferably comprising contiguous nucleotides, of at least about 6 nucleotides to about 60 nucleotides, preferably at least about 8 to 10 nucleotides in length, more preferably at least about 12 nucleotides in length e.g., about 15 to 35 nucleotides, or about 15 to 25 nucleotides, 18 to 20 nucleotides, or about 20 to 35 nucleotides, which can be typically used in PCR amplification assays, hybridization assays, or in microarrays. It will be understood that the term “oligonucleotide” is substantially equivalent to the terms primer, probe, or amplimer, as commonly defined in the art. It will also be appreciated by those skilled in the pertinent art that a longer oligonucleotide probe, or mixtures of probes, e.g., degenerate probes, can be used to detect longer, or more complex, nucleic acid sequences, for example, genomic DNA. In such cases, the probe may comprise at least 20-200 nucleotides, preferably, at least 30-100 nucleotides, more preferably, 50-100 nucleotides.

The term “marker” in the context of the present invention refers to a nucleic acid fragment, a peptide, or a polypeptide, which is differentially present in a sample taken from individuals or patients who are non-steroid responders as compared to a comparable sample taken from subjects who steroid responders.

As used herein the phrase “differentially present” refers to differences in the quantity of a marker present in a sample taken from steroid responders as compared to a comparable sample taken from non-steroid responders. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present. One of ordinary skill in the art could easily determine such relative levels of the markers.

As used herein, the term “level” refers to expression levels of RNA and/or protein or to DNA copy number of a marker of the present invention. Typically the level of the marker in a biological sample obtained from the subject is different (i.e., increased or decreased) from the level of the same variant in a similar sample obtained from a healthy individual (examples of biological samples are described herein). Determining the level of the same variant in normal tissues of the same origin is preferably effected along-side to detect an elevated expression and/or amplification and/or a decreased expression, of the variant as opposed to the normal tissues.

A “test amount” of a marker refers to an amount of a marker present in a sample being tested. A test amount can be either in absolute amount (e.g., microgram/nal) or a relative amount (e.g., relative intensity of signals). A “control amount” of a marker can be any amount or a range of amounts to be compared against a test amount of a marker. A control amount can be either in absolute amount (e.g., microgram/nal) or a relative amount (e.g., relative intensity of signals).

“Detect” refers to identifying the presence, absence or amount of the object to be detected.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above. Diagnosis of a disease according to the present invention can be effected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

The term “sample” is meant to be interpreted in its broadest sense. A “sample” refers to a biological sample, such as, for example; one or more cells, tissues, or fluids (including, without limitation, plasma, serum, whole blood, cerebrospinal fluid, lymph, tears, urine, saliva, milk, pus, and tissue exudates and secretions) isolated from an individual or from cell culture constituents, as well as samples obtained from, for example, a laboratory procedure. A biological sample may comprise chromosomes isolated from cells (e.g., a spread of metaphase chromosomes), organelles or membranes isolated from cells, whole cells or tissues, nucleic acid such as genomic DNA in solution or bound to a solid support such as for Southern analysis, RNA in solution or bound to a solid support such as for Northern analysis, cDNA in solution or bound to a solid support, oligonucleotides in solution or bound to a solid support, polypeptides or peptides in solution or bound to a solid support, a tissue, a tissue print and the like.

Numerous well known tissue or fluid collection methods can be utilized to collect the biological sample from the subject in order to determine the level of DNA, RNA and/or polypeptide of the variant of interest in the subject. Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

“Microarray” is an array of distinct polynucleotides, oligonucleotides, polypeptides, peptides, or antibodies affixed to a substrate, such as paper, nylon, or other type of membrane; filter; chip; glass slide; or any other type of suitable support.

As used herein, the term “linker” means a chemical moiety which covalently joins the reactive groups already on the substrate and the molecule (e.g., DNA, antibody, or polypeptide) to be eventually immobilized, having a backbone of chemical bonds forming a continuous connection between the reactive groups on the substrate and the binding elements, and having a plurality of freely rotating bonds along that backbone.

### Biomarkers

Identification of steroid responders prior to treatment, allows the use of steroids to become much safer. Further, the ability to stratify a patient population into responders and non-responders would be of great benefit to the design of clinical trials on corticosteroid drugs for use, for example, in the eye.

Without wishing to be bound by theory, it was hypothesized that genetic variability in the glucocorticoid response or metabolism pathway associates with steroid response. One of the candidate genes initially selected for study was the glucocorticoid receptor gene (GCR) (5q31) which was hypothesized to be a primary mediator of steroid response. A detailed description of the methodologies used are described in the examples section which follows. Briefly, the steps were as follows: phenotyping and genotyping of a cohort of patients; association of magnitude of IOP change as a continuous variable; candidate gene analysis, for example, glucocorticoid receptor polymorphisms; pathway analysis; whole genome screen. The results showed that: 48 different SNPs within 33 genes were found to correlate (p<0.001) with magnitude of ΔIOP following IVTA. The strongest association involves a SNP within an as-yet poorly described G-protein coupled receptor (p=3.05×10⁻⁸). Four individual SNPs within a single transporter gene were identified (p between 5.59×10⁻⁴ and 2.81×10⁻⁵). Other genes with multiple SNPs included a translation elongation factor, an F-box protein, an oxysterol binding protein, and a solute carrier family gene.

The glucocorticoid receptor is encoded by the gene GR. There are at least 24 polymorphisms in the human GR gene. Some examples of such polymorphisms include: 1. ER22/23EK (HapMap rs6190), a GAGAGG→GAAAAG substitution, which results in a GluArg→GluLys (ER→EK) substitution at codons 22-23 (incidence 7% heterozygote); 2. N363S (HapMap rs56149945), an AAT→AGT substitution, which results in an Asn→Ser (N*S) substitution at codon 363 (incidence 6% heterozygote); 3. Bell (HapMap rs62375508), a C→G substitution in intron 2, 646 nt downstream from exon 2 (incidence 48% heterozygote, 12% homozygote); 4. N766N (HapMap rs6196), an AAT→AAC substitution, which results in an Asn→Asn substitution (N→N) at codon 766 (incidence 23% homozygote, 10% homozygote); 5. A G→C substitution within intron 3 (HapMap rs61753484), 46 nt upstream from exon 4 (incidence 3% heterozygote); and, 6. A G→T substitution within intron 4 (HapMap rs6188), nt upstream from exon 5 (incidence 42% heterozygote, 10% homozygote).

The inventors have discovered, that biomarkers corresponding to the nucleic acid molecules listed in Tables 1 to 3 comprising one or more polymorphisms are important in predicting, identifying and diagnosing individuals who may or may not be responsive to steroid treatments; or, in cases where individuals will have an adverse reaction to steroid treatment, such as for example, an increase in intraocular pressure. The biomarkers identified, see, for example, Tables 2 and 3, comprise nucleotide polymorphisms which indicate the likelihood of risk associated with steroid treatments. These SNPs serve as markers for risk of a steroid response (secondary glaucoma induced by corticosteroids) and can also be used to differentiate between a steroid responder or non-responder, or identify those individuals who will have an adverse reaction to the steroid treatment. Some of these molecules, e.g. the genes of the nucleic acid molecules illustrated in Table 3, can be also differentially expressed (or altered, e.g. single nucleotide polymorphisms). Accordingly, the molecules, identified by their NCBI RefSeq accession numbers can be used, inter alfa, in molecular medicine applications and as targets for identifying new drugs and therapies.

In a preferred embodiment, a biomarker comprises at least one of: NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6VOD2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); or, NM_{—}002697 (POU2F1, POU domain, class 2, transcription factor 1), a G-protein coupled receptor, a translation elongation factor, an F-box protein, an oxysterol binding protein, a solute carrier family gene, variants, mutants, homologs, alleles and fragments, or complementary sequences thereof.

In a preferred embodiment, the markers comprise at least one single nucleotide polymorphisms. The polymorphisms may be consecutive or dispersed along the molecule.

In another preferred embodiment, the biomarkers further comprise glucocorticoid receptor polymorphisms and single nucleotide polymorphisms in BclI, N766N and intron 4.

According to these embodiment, the biomarkers relate to genes, mRNAs, and proteins corresponding to the biomarkers as described in Tables 1 to 3. A biomarker can be a specific nucleic acid molecule listed in Tables 1 to 3, single nucleotide polymorphisms of nucleic acid molecules listed in Tables 1 to 3, alternative splice variants of the gene, fragments of genomic DNA comprising the gene (or a fragment thereof), mRNA molecules corresponding to the gene (or fragments thereof), cDNA corresponding to the gene (or fragments thereof), protein corresponding to the gene (or fragments thereof), and the like. The set of biomarkers can be assessed according to the invention by a variety of methods. Such methods of characterizing whether a responder vs. a non-responder has a biomarker signature according to the invention, include, but are not limited to, single nucleotide polymorphisms, DNA copy number analysis of one or more genomic regions having the genes as listed in Tables 1 to 3, RNA expression analysis of the one or more genes as listed in Table 1, and detection of proteins expressed from the one or more genes of the nucleic acid molecules as listed in Tables 1 to 3. In one aspect of this embodiment, a composition (e.g., kit or array) is provided which comprises a set of probes capable of detecting at least 1, 2, 3, 4, 5, 6, 7, 8, 9, 10, 11, 12, 13, 14, 15, 16, 17, 18, 19, 20, 25, 30, or 35 of the biomarkers listed in Tables 1 to 3.

In a preferred embodiment, detection of at least one single nucleotide polymorphism in one or more biomarkers comprising: NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6V0D2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4, a G-protein coupled receptor, a translation elongation factor, an F-box protein, an oxysterol binding protein, a solute carrier family gene, variants, mutants, homologs, alleles and fragments, or sequences complementary thereto, is predictive of whether a patient will respond to steroid treatment and whether there will be a rise in, for example, intraocular pressure.

In another preferred embodiment, the biomarker comprises at least one encoded product from genes of: NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6V0D2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4, a G-protein coupled receptor, a translation elongation factor, an F-box protein, an oxysterol binding protein, a solute carrier family gene, variants, mutants, homologs, alleles and fragments, or complementary sequences thereof.

In a preferred embodiment, the markers comprise at least one single nucleotide polymorphisms. The polymorphisms may be consecutive or dispersed along the molecule.

Any polynucleotide that encodes an amino acid sequence of a biomarker polypeptide or peptide, or complementary sequences, or related fragments or variants, is included in the invention. Polynucleotide variants of the present invention include, but are not limited to, variants that share at least 40%, 50%, 60%, 70%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% nucleotide sequence identity with any one of the sequences of NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6VOD2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4, a G-protein coupled receptor, a translation elongation factor, an F-box protein, an oxysterol binding protein, a solute carrier family gene, variants, mutants, homologs, alleles and fragments, or sequences complementary thereto.

In various embodiments, the invention encompasses polynucleotide fragments, which include, but are not limited to, fragments comprising at least 8, 10, 12, 15, 18, 20, 25, 30, 35, 36, 40, 45 contiguous nucleotides of any one of the sequences of NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6VOD2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4, a G-protein coupled receptor, a translation elongation factor, an F-box protein, an oxysterol binding protein, a solute carrier family gene, variants, mutants, homologs, alleles and fragments, or sequences complementary thereto.

In a preferred embodiment, the markers comprise at least one single nucleotide polymorphisms. The polymorphisms may be consecutive or dispersed along the molecule.

In one embodiment, the invention provides a set of DNA copy number or sequence biomarkers. According to this embodiment, the biomarkers relate to genomic DNA regions corresponding to the biomarkers as described in Tables 1 to 3. The biomarker, according to this embodiment, can be a genomic region, marker, loci, or the such, comprising a specific single nucleotide polymorphisms of nucleic acid molecules listed in Table 1 to 3.

In one embodiment, the invention provides a set of mRNA biomarkers. According to this embodiment, the biomarkers relate to mRNAs corresponding to the biomarkers as described in Tables 1 to 3. In one aspect of this embodiment, a steroid non-responder versus a steroid responder is classified by measuring and/or sequencing at least 1, 2, 3, 4, 5, 6, 7, 8, 9, 10, 11, 12, 13, 14, 15, 16, 17, 18, 19, 20, 25, 30, or 35 mRNA biomarkers corresponding to the nucleic acid molecules listed in Table 1 to 3. In one aspect of this embodiment classification can be by measuring and/or sequencing a set of at least 1, 2, 3, 4, 5, 6, 7, 8, 9, 10, 11, 12, 13, 14, 15, 16, 17, 18, 19, 20, 25, 30, or 35 mRNAs corresponding to the biomarkers listed in Table 1 to 3, and comparing the measured values and/or sequences to a reference (or control). Such methods of characterizing whether a cancer has a biomarker signature according to the invention, include, but are not limited to, microarray based mRNA expression analysis or quantitative PCR analysis of one or more transcripts (of fragments thereof) corresponding to the genes as listed in Table 1 to 3. In one aspect of this embodiment, a composition (e.g., kit or array) is provided which comprises a set of probes capable of detecting at least 1, 2, 3, 4, 5, 6, 7, 8, 9, 10, 11, 12, 13, 14, 15, 16, 17, 18, 19, 20, 25, 30, or 35 of the mRNAs (or fragments thereof) corresponding to the nucleic acid molecules listed in Tables 1 to 3.

Also encompassed by the invention are polynucleotides that are capable of hybridizing to the nucleotide sequences of NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6VOD2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4, variants, mutants, homologs, alleles and fragments, or sequences complementary thereto, under various conditions of stringency.

In a preferred embodiment, the nucleotides comprise at least one single nucleotide polymorphisms.

Hybridization conditions are typically based on the melting temperature (Tₘ) of the nucleic acid binding complex or probe (see, G. M. Wahl and S. L. Berger, 1987; Methods Enzymol., 152:399-407 and A. R. Kimmel, 1987; Methods of Enzymol., 152:507-511), and may be used at a defined stringency. As a non-limiting example, moderate stringency conditions comprise a prewashing solution of 2×SSC, 0.5% SDS, 1.0 mM EDTA, pH 8.0, and hybridization conditions of 50° C., 5×SSC, and overnight incubation.

As will be appreciated by the skilled practitioner in the art, the degeneracy of the genetic code results in the production of a multitude of nucleotide sequences encoding a biomarker polypeptide. Some of the degenerate sequences may bear minimal homology to the nucleotide sequences of the biomarkers. Accordingly, the present invention contemplates each and every possible variation of nucleotide sequence that could be made by selecting combinations based on possible codon choices. These combinations are made in accordance with the standard triplet genetic code as applied to the nucleotide sequence of the originally identified biomarker polypeptides, and all such variations are to be considered as being specifically disclosed.

For some purposes, it may be advantageous to produce polynucleotides encoding a biomarker polypeptide, or its fragments, variants, or derivatives, which possess a substantially different codon usage. Codons may be selected to increase the rate at which expression of the peptide/polypeptide occurs in a particular prokaryotic or eukaryotic host in accordance with the frequency with which particular codons are utilized by the host. Alterations in codon usage can also be used to produce RNA transcripts having more desirable properties, such as a greater half-life, than transcripts produced from the naturally occurring sequence. In particular, RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl, rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytosine, guanine, thymine, and uridine which are not as easily recognized by endogenous endonucleases.

The biomarker polynucleotides, and complementary sequences, and fragments thereof, can be engineered using methods generally known in the art in order to alter the sequences for a variety of reasons, including, but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and the like.

Also encompassed by the invention derivatives of the biomarker polynucleotides, and complementary sequences, and fragments thereof, which comprise one or more chemical modification, for example, replacement of hydrogen by an alkyl, acyl, or amino group. Alternatively, derivative polynucleotides may comprise non-naturally-occurring portions, such as altered sugar moieties or inter-sugar linkages. Exemplary among these are phosphorothioate and other sulfur containing species which are known in the art. Derivative polynucleotides may also contain detection labels, including radionucleotides (e.g., ³²P, ³H, and ³⁵S), enzymes, fluorescent (e.g., rhodamine, fluorescein, and Cy3, Cy5), chemiluminescent, or chromogenic, and other labels (e.g., DNP, digoxigenin, and biotin) such as substrates, cofactors, inhibitors, magnetic particles, and the like.

A wide variety of labels and conjugation techniques are known and employed by those skilled in the art. Nucleic acid labeling can be achieved by oligo-labeling, nick translation, end-labeling, or PCR amplification using a labeled primer. Alternatively, polynucleotides, or any portions or fragments thereof, may be cloned into a vector for the production of labeled mRNA sequences. Such vectors are known in the art, are commercially available, and may be used to synthesize labeled RNA in vitro by addition of an appropriate RNA polymerase, such as T7, T3, or SP(6) and labeled nucleotides. These procedures may be conducted using a variety of commercially available kits (e.g., from Amersham-Pharmacia; Promega Corp.; and U.S. Biochemical Corp., Cleveland, Ohio).

The present invention also encompasses the production of polynucleotides, or portions thereof, which encode a biomarker polypeptide, see, for example, Tables 1-3, and its fragments, and derivatives, by any means. For example, synthetic chemistry (see, for example, M. H. Caruthers et al., 1980, Nucl. Acids Res. Symp. Ser., 215-223 and T. Horn et al., 1980, Nucl. Acids Res. Symp. Ser., 225-232). After production, the synthetic sequence may be inserted into any of the many available expression vectors and cell systems using reagents that are well known to those in the art. Moreover, synthetic chemistry may be used to introduce mutations into a sequence encoding a biomarker polypeptide, or any fragment, variant, or derivative thereof. Alternatively, the polynucleotides of the invention can be produced by PCR amplification of the cloned sequences. In addition, the polynucleotides may be produced by recombinant systems, including cell-based and cell-free systems.

Polynucleotides that encode a biomarker polypeptide, or fragments, variants, or derivatives thereof, may be used in recombinant DNA molecules to direct the expression of a biomarker, or fragments or functional equivalents thereof, in appropriate host cells. Because of the inherent degeneracy of the genetic code, other DNA sequences, which encode substantially the same or a functionally equivalent amino acid sequence, may be produced and these sequences may be used to clone and express a biomarker polypeptide. For expression in recombinant systems, a start and stop codons may be added to the nucleic acid sequence of a biomarker polypeptide. In addition, nucleotide sequences encoding epitopes or protein tags can be added to the nucleic acid sequence of the biomarker polypeptide, as described in detail herein. Methods of cloning and expression are well known to those skilled in the art and are described in numerous publication's, for example, Sambrook, Fritsch, and Maniatis, Molecular Cloning: a Laboratory Manual, 2^{nd} Edition, Cold Spring Harbor Laboratory Press, USA, (1989).

Polypeptides:

In one embodiment, detection of gene products in a sample comprising at least one gene product of: NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6V0D2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4, peptides, fragments, variants, and derivatives thereof, that can be used to predict and differentiate between a steroid responder versus non-responder.

In another preferred embodiment, the markers comprise at least one single nucleotide polymorphism.

In another preferred embodiment, a composition comprises an antibody specific for a gene product of single nucleotide polymorphism, mutants and variants thereof comprising: any one molecule in Tables 1 to 3.

In another preferred embodiment, the polynucleotides comprising single nucleotide polymorphisms of, for example, the genes identified in Table 3, encode a polypeptide biomarker.

The protein biomarkers, according to this embodiment, can be any protein (or fragments thereof) that correspond to one or more of the genes corresponding to the nucleic acid molecules in Tables 1 to 3. The set of protein biomarkers can be assessed according to the invention by a variety of methods capable of ascertaining protein expression levels of a particular protein. Such methods include, but are not limited to, monoclonal or polyclonal antibody based detection (via IHC, ELISA, or other suitable method) of proteins expressed from the one or more genes from corresponding to the nucleic acid molecules in Tables 1 to 3.

In accordance with the invention, biomarker peptides can range in size from 5 amino acid residues to all but one residue of the entire sequence. Accordingly, peptides include, but are not limited to, fragments comprising at least 5, 6, 7, 8, 9, 10, 11, 12, 13, 14, or 15 contiguous amino acids of any one of: NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6VOD2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4, fragments or variants thereof.

The biomarker polypeptides, peptides, or fragments or variants thereof, may be linked to short tags, e.g., epitope tags such as HA and the like, or to other proteins, such as GST, GFP (e.g., GFP Y66F, GFP Y66H, GFP Y66W, wild type GFP, GFP S65A, GFP S65L, GFP S65T, ECFP, EYFP, DsRed; BD Biosciences CLONTECH, Palo Alto, Calif.), thioredoxin, maltose binding protein, etc. Also provided by the invention are chemically modified derivatives of the peptides and polypeptides of the invention that may provide additional advantages such as increased solubility, stability, and circulating time of the polypeptide. The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

In addition, amino acid sequence variants of the present invention include, but are not limited to, variants that share at least 40%, 50%, 60%, 61%, 67%, 70%, 74%, 76%, 80%, 81%, 84%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% nucleotide sequence identity with any one of NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6V0D2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in Bell, N766N and/or intron 4, variants, or fragments of.

Polypeptide and peptide variants include variants differing by the addition, deletion, or substitution of one or more amino acid residues. For example, to isolate biomarker polypeptides or peptides, it may be useful to encode a tagged biomarker peptide or polypeptide that can be recognized by a commercially available antibody. In particular, a peptide or polypeptide can be fused or linked to epitope tags (e.g., FLAG, HA, GST, thioredoxin, maltose binding protein, etc.), or affinity tags such as biotin and/or streptavidin. As one example, a system for the ready purification of non-denatured fusion proteins expressed in human cell lines has been described by Janknecht et al., (1991, Proc. Natl. Acad. Sci. USA, 88:8972-8976). In this system, the gene of interest is subcloned into a vaccinia recombination plasmid such that the open reading frame of the gene is translationally fused to an amino-terminal tag having six histidine residues. The tag serves as a matrix-binding domain for the fusion protein. Extracts from cells infected with the recombinant vaccinia virus are loaded onto an Ni²⁺ nitriloacetic acid-agarose column and histidine-tagged proteins are selectively eluted with imidazole-containing buffers.

A peptide or polypeptide tagged with an epitope or protein may also be engineered to contain a cleavage site located between the binder coding sequence and the tag coding sequence. This can be used to remove the tag, and isolate the biomarker peptide or polypeptide. The biomarker peptides or polypeptides of the invention can be covalently attached to chemical moieties via the amino acid backbone. For these purposes, the peptides or polypeptides may be modified by N- or C-terminal processing of the sequences (e.g., proteolytic processing), deletion of the N-terminal methionine residue, etc. The polypeptides may also be modified with a detectable label, such as an enzymatic, fluorescent, isotopic or affinity label to allow for detection and isolation of the protein, as described in detail herein.

Also included are modified polypeptides and peptides in which one or more residues are modified, and mutants comprising one or more modified residues. Amino acid variants of the invention can be generated by employing the techniques of gene-shuffling, motif-shuffling, exon-shuffling, and/or codon-shuffling (collectively referred to as “DNA shuffling”). DNA shuffling can be employed to generate peptides or polypeptides with altered activity. See, generally, U.S. Pat. Nos. 5,605,793; 5,811,238; 5,830,721; 5,834,252; and 5,837,458, and Patten et al., 1997, Curr. Opinion Biotechnol., 8:724-33; Harayama, 1998, Trends Biotechnol., 16(2):76-82; Hansson, et al., 1999, J. Mol. Biol., 287:265-76; and Lorenzo and Blasco, 1998, Biotechniques, 24(2):308-313, the contents of each of which are hereby incorporated by reference in its entirety.

In one embodiment of the invention, alteration of one or more of the biomarker polypeptide sequences can be achieved by DNA shuffling. DNA shuffling involves the assembly of two or more DNA segments by homologous or site-specific recombination to generate variation in the protein-coding sequence. In another embodiment, the encoded peptides or polypeptides, may be altered by subjecting the coding sequences error-prone PCR, random nucleotide insertion, or other methods, prior to recombination. In another embodiment, one or more components, motifs, sections, parts, domains, fragments, etc., of a polynucleotide encoding a peptide or polypeptide of this invention may be recombined with one or more components, motifs, sections, parts, domains, fragments, etc. of one or more heterologous molecules.

The peptides and polypeptides may be differentially modified during or after translation, e.g., by derivatization with known protecting/blocking groups, proteolytic cleavage, linkage to an antibody molecule or other cellular ligand, etc. Useful modifications may include glycosylation, amidation, phosphorylation, sulfation, reduction/alkylation (Tarr, 1986, Methods of Protein Microcharacterization, J. E. Silver, Ed., Humana Press, Clifton, N.J., pp. 155-194); acylation (Tarr, supra); chemical coupling (Mishell and Shiigi (Eds), 1980, Selected Methods in Cellular Immunology, W H Freeman, San Francisco, Calif.; U.S. Pat. No. 4,939,239); and mild formalin treatment (Marsh, 1971, Int. Arch. of Allergy and Appl. Immunol. 41:199-215). Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; etc. Additional post-translational modifications encompassed by the invention include, for example, e.g., attachment of N-linked or O-linked carbohydrate chains, processing of N-terminal or C-terminal ends, chemical modifications of N-linked or O-linked carbohydrate chains, and addition or deletion of an N-terminal methionine residue as a result of prokaryotic host cell expression.

Additionally, D-amino acids, non-natural amino acids, or non-amino acid analogs can be substituted or added to produce a modified polypeptide. Furthermore, the polypeptides disclosed herein can be modified using polyethylene glycol (PEG) according to known methods (S. I. Wie et al., 1981, Int. Arch. Allergy Appl. Immunol. 64(1):84-99) to produce a protein conjugated with PEG. In addition, PEG can be added during chemical synthesis of the protein. Modifications or sequence variations may occur at the amino- or carboxy-terminal positions of the reference polypeptide sequence or anywhere between those terminal positions, interspersed either individually among the amino acids in the reference sequence or in one or more contiguous groups within the reference sequence. The polypeptides and peptides of this invention can be isolated, synthetic, or recombinant. The amino acid sequences may be obtained as individual polypeptides or peptides, or part of a complex.

Polypeptides or peptides may also be modified with a label capable of providing a detectable signal, either directly or indirectly, including, but not limited to, radioisotope, fluorescent, and enzyme labels. Fluorescent labels include, for example, Coumarin (e.g., Hydroxycoumarin, Aminocoumarin, Methoxycoumarin), R-Phycoerythrin (PE), Fluorescein, FITC, Fluor X, DTAF, Auramine, Alexa (e.g., ALEXA FLUOR 350, -430, -488, -532, -546, -555, -568, -594, -633, -647, -660, -680, -700, -750), BODIPY-FL, Sulforhodamine (e.g., Texas Red®), Carbocyanine (e.g., Cy2, Cy3, Cy3.5, Cy5, Cy5.5, Cy7), Rhodamine, XRITC, TRITC, Lissamine Rhodamine B, Peridinin Chlorphyll Protein (PerCP), Allophycocyanin (APC), PE-Cy5 conjugates (e.g., Cychrome, TRI-COLOR™, QUANTUM RED™), PE-Cy5.5 conjugates, PE-Cy7 conjugates, PE-Texas Red conjugates (e.g., Red613), PC5-PE-Cy5 conjugates, PerCP-Cy5.5 conjugates (e.g., TruRed), APC-Cy5.5 conjugates, APC-Cy7 conjugates, ECD-PE-Texas Red conjugates, Sulfonated Pyrene (e.g., Cascade Blue), AMCA Blue, Lucifer Yellow.

Preferred isotope labels include ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. Preferred enzyme labels include peroxidase, β-glucuronidase, β-D-glucosidase, β-D-galactosidase, urease, glucose oxidase plus peroxidase, and alkaline phosphatase (see, e.g., U.S. Pat. Nos. 3,654,090; 3,850,752 and 4,016,043). Enzymes can be conjugated by reaction with bridging molecules such as carbodiimides, diisocyanates, glutaraldehyde, and the like. Enzyme labels can be detected visually, or measured by calorimetric, spectrophotometric, fluorospectrophotometric, amperometric, or gasometric techniques. Other labeling systems, such as avidin/biotin, colloidal gold (e.g., NANOGOLD™), Tyramide Signal Amplification (TSA™), are known in the art, and are commercially available (see, e.g., ABC kit, Vector Laboratories, Inc., Burlingame, Calif.; NEN™ Life Science Products, Inc., Boston, Mass.; Nanoprobes, Inc., 95 Horse Block Road, Yaphank, N.Y.).

Biomarker polypeptides (e.g., encoded products of NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6V0D2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4), peptides, and fragments, variants, and derivatives thereof, may be produced by direct peptide synthesis using solid-phase techniques (J. Merrifield, 1963, J. Am. Chem. Soc., 85:2149-2154; J. Y. Roberge et al., 1995, Science, 269:202-204). Protein or peptide synthesis may be performed using manual techniques or by automation. Automated synthesis may be achieved, for example, using ABI 431A Peptide Synthesizer (PE Biosystems). Various fragments of a biomarker polypeptide or peptide can be chemically synthesized separately and then combined using chemical methods to produce the full-length molecule. The newly synthesized peptide can be substantially purified by preparative high performance liquid chromatography (e.g., T. Creighton, 1983, Proteins, Structures and Molecular Principles, W. H. Freeman and Co., New York, N.Y.), by reversed-phase high performance liquid chromatography, or other purification methods as are known in the art. The composition of the synthetic peptides may be confirmed by amino acid analysis or sequencing (e.g., the Edman degradation procedure; Creighton, supra). In addition, the amino acid sequence of biomarker peptide or polypeptide or any portion thereof, may be altered during direct synthesis and/or combined using chemical methods with sequences from other proteins, or any part thereof, to produce a variant peptide or polypeptide.

### Microarrays

The biomarkers of the invention can also be identified, confirmed, and/or measured using the microarray technique. Thus, the expression profile biomarkers can be measured in either fresh or paraffin-embedded tissue, other biological samples, using microarray technology. In this method, polynucleotide sequences of interest are plated, or arrayed, on a microchip substrate. The arrayed sequences are then hybridized with specific probes from cells or tissues of interest. As with the RT-PCR method, the source of mRNA typically is total RNA isolated from human tumors or tumor cell lines, and corresponding normal tissues or cell lines. Thus RNA can be isolated from a variety of samples. The examples section which follows, provides a detailed description of the methods used.

In one embodiment of the microarray technique, PCR amplified inserts of cDNA clones are applied to a substrate in a dense array. In one aspect, at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al. (1996) Proc. Natl. Acad. Sci. USA 93(2):106-149) or detection of SNPs. Microarray analysis can also be performed by commercially available equipment, following manufacturer's protocols, such as by using the Affymetrix GenChip technology, or Incyte's microarray technology.

The development of microarray methods for large-scale analysis of gene expression makes it possible to search systematically for molecular markers of cancer classification and outcome prediction in a variety of tumor types.

### Data and Analysis

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention relates to embodiments that include methods for providing genetic information over networks such as the Internet.

### Methods for Analyzing Other Genes/Biomarkers

The present invention also provides a method for genotyping one or more genes (and/or biomarkers in Tables 1 to 3) by determining whether an individual has one or more nucleotide variants (or amino acid variants) in one or more of the genes (or proteins). Genotyping one or more genes according to the methods of the invention in some embodiments, can provide more evidence for determining therapy, diagnosis, and prognosis.

The genes (and/or biomarkers in Tables 1 to 3) of the invention can be analyzed by any method useful for determining alterations in nucleic acids or the proteins they encode. According to one embodiment, the ordinary skilled artisan can analyze the one or more genes for mutations including deletion mutants, insertion mutants, frameshift mutants, nonsense mutants, missense mutant, and splice mutants.

Nucleic acid used for analysis of the one or more genes (and/or biomarkers from Tables 1 to 3) can be isolated from cells in the sample according to standard methodologies (Sambrook et al., 1989). The nucleic acid, for example, may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified. Depending on the format of the assay for analyzing the one or more tumors suppressor genes, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Various types of defects can occur in the genes (and/or biomarkers of Tables 1 to 3) of the invention. Thus, “alterations” should be read as including deletions, insertions, point mutations, and duplications. Point mutations result in stop codons, frameshift mutations or amino acid substitutions. Mutations in and outside the coding region of the one or more genes may occur and can be analyzed according to the methods of the invention.

Similarly, a method for haplotyping one or more genes is also provided. Haplotyping can be done by any methods known in the art. For example, only one copy of one or more genes can be isolated from an individual and the nucleotide at each of the variant positions is determined. Alternatively, an allele specific PCR or a similar method can be used to amplify only one copy of the one or more genes in an individual, and the SNPs at the variant positions of the present invention are determined. The Clark method known in the art can also be employed for haplotyping. A high throughput molecular haplotyping method is also disclosed in Tost et al., Nucleic Acids Res., 30(19):e96 (2002), which is incorporated herein by reference.

Thus, additional variant(s) that are in linkage disequilibrium with the variants and/or haplotypes of the present invention can be identified by a haplotyping method known in the art, as will be apparent to a skilled artisan in the field of genetics and haplotyping. The additional variants that are in linkage disequilibrium with a variant or haplotype of the present invention can also be useful in the various applications as described below.

For purposes of genotyping and haplotyping, both genomic DNA and mRNA/cDNA can be used, and both are herein referred to generically as “gene.”

Numerous techniques for detecting nucleotide variants are known in the art and can all be used for the method of this invention. The techniques can be protein-based or nucleic acid-based. In either case, the techniques used must be sufficiently sensitive so as to accurately detect the small nucleotide or amino acid variations. Very often, a probe is utilized which is labeled with a detectable marker. Unless otherwise specified in a particular technique described below, any suitable marker known in the art can be used, including but not limited to, radioactive isotopes, fluorescent compounds, biotin which is detectable using streptavidin, enzymes (e.g., alkaline phosphatase), substrates of an enzyme, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977).

In a nucleic acid-based detection method, target DNA sample, i.e., a sample containing genomic DNA, cDNA, and/or mRNA, corresponding to the one or more genes must be obtained from the individual to be tested. Any tissue or cell sample containing the genomic DNA, mRNA, and/or cDNA (or a portion thereof) corresponding to the one or more genes can be used. For this purpose, a tissue sample containing cell nucleus and thus genomic DNA, or cells etc, can be obtained from the individual. Blood samples can also be useful except that only white blood cells and other lymphocytes have cell nucleus, while red blood cells contain only mRNA. Nevertheless, mRNA is also useful as it can be analyzed for the presence of nucleotide variants in its sequence or serve as template for cDNA synthesis. The tissue or cell samples can be analyzed directly without much processing. Alternatively, nucleic acids including the target sequence can be extracted, purified, and/or amplified before they are subject to the various detecting procedures discussed below. Other than tissue or cell samples, cDNAs or genomic DNAs from a cDNA or genomic DNA library constructed using a tissue or cell sample obtained from the individual to be tested are also useful.

To determine the presence or absence of a particular nucleotide variant, one technique is to sequence the target genomic DNA or cDNA, particularly the region encompassing the nucleotide variant locus to be detected. Various sequencing techniques are generally known and widely used in the art including the Sanger method and Gilbert chemical method. The pyrosequencing method monitors DNA synthesis in real time using a luminometric detection system. Pyrosequencing has been shown to be effective in analyzing genetic polymorphisms such as single-nucleotide polymorphisms and thus can also be used in the present invention. See Nordstrom et al., Biotechnol. Appl. Biochem., 31(2):107-112 (2000); Ahmadian et al., Anal. Biochem., 280:103-110 (2000).

Alternatively, the restriction fragment length polymorphism (RFLP) and AFLP method may also prove to be useful techniques. In particular, if a nucleotide variant in the target DNA corresponding to the one or more genes results in the elimination or creation of a restriction enzyme recognition site, then digestion of the target DNA with that particular restriction enzyme will generate an altered restriction fragment length pattern. Thus, a detected RFLP or AFLP will indicate the presence of a particular nucleotide variant.

Another useful approach is the single-stranded conformation polymorphism assay (SSCA), which is based on the altered mobility of a single-stranded target DNA spanning the nucleotide variant of interest. A single nucleotide change in the target sequence can result in different intramolecular base pairing pattern, and thus different secondary structure of the single-stranded DNA, which can be detected in a non-denaturing gel. See Orita et al., Proc. Natl. Acad. Sci. USA, 86:2776-2770 (1989). Denaturing gel-based techniques such as clamped denaturing gel electrophoresis (CDGE) and denaturing gradient gel electrophoresis (DGGE) detect differences in migration rates of mutant sequences as compared to wild-type sequences in denaturing gel. See Miller et al., Biotechniques, 5:1016-24 (1999); Sheffield et al., Am. J. Hum, Genet., 49:699-706 (1991); Wartell et al., Nucleic Acids Res., 18:2699-2705 (1990); and Sheffield et al., Proc. Natl. Acad. Sci. USA, 86:232-236 (1989). In addition, the double-strand conformation analysis (DSCA) can also be useful in the present invention. See Arguello et al., Nat. Genet., 18:192-194 (1998).

The presence or absence of a nucleotide variant at a particular locus in the one or more genes of an individual can also be detected using the amplification refractory mutation system (ARMS) technique. See e.g., European Patent No. 0,332,435; Newton et al., Nucleic Acids Res., 17:2503-2515 (1989); Fox et al., Br. J. Cancer, 77:1267-1274 (1998); Robertson et al., Eur. Respir. J., 12:477-482 (1998). In the ARMS method, a primer is synthesized matching the nucleotide sequence immediately 5′ upstream from the locus being tested except that the 3′-end nucleotide which corresponds to the nucleotide at the locus is a predetermined nucleotide. For example, the 3′-end nucleotide can be the same as that in the mutated locus. The primer can be of any suitable length so long as it hybridizes to the target DNA under stringent conditions only when its 3′-end nucleotide matches the nucleotide at the locus being tested. Preferably the primer has at least 12 nucleotides, more preferably from about 18 to 50 nucleotides. If the individual tested has a mutation at the locus and the nucleotide therein matches the 3′-end nucleotide of the primer, then the primer can be further extended upon hybridizing to the target DNA template, and the primer can initiate a PCR amplification reaction in conjunction with another suitable PCR primer. In contrast, if the nucleotide at the locus is of wild type, then primer extension cannot be achieved. Various forms of ARMS techniques developed in the past few years can be used. See e.g., Gibson et al., Clin. Chem. 43:1336-1341 (1997).

Similar to the ARMS technique is the mini sequencing or single nucleotide primer extension method, which is based on the incorporation of a single nucleotide. An oligonucleotide primer matching the nucleotide sequence immediately 5′ to the locus being tested is hybridized to the target DNA or mRNA in the presence of labeled dideoxyribonucleotides. A labeled nucleotide is incorporated or linked to the primer only when the dideoxyribonucleotides matches the nucleotide at the variant locus being detected. Thus, the identity of the nucleotide at the variant locus can be revealed based on the detection label attached to the incorporated dideoxyribonucleotides. See Syvanen et al., Genomics, 8:684-692 (1990); Shumaker et al., Hum. Mutat., 7:346-354 (1996); Chen et al., Genome Res., 10:549-547 (2000).

Another set of techniques useful in the present invention is the so-called “oligonucleotide ligation assay” (OLA) in which differentiation between a wild-type locus and a mutation is based on the ability of two oligonucleotides to anneal adjacent to each other on the target DNA molecule allowing the two oligonucleotides joined together by a DNA ligase. See Landergren et al., Science, 241:1077-1080 (1988); Chen et al, Genome Res., 8:549-556 (1998); Iannone et al., Cytometry, 39:131-140 (2000). Thus, for example, to detect a single-nucleotide mutation at a particular locus in the one or more genes, two oligonucleotides can be synthesized, one having the sequence just 5′ upstream from the locus with its 3′ end nucleotide being identical to the nucleotide in the variant locus of the particular gene, the other having a nucleotide sequence matching the sequence immediately 3′ downstream from the locus in the gene. The oligonucleotides can be labeled for the purpose of detection. Upon hybridizing to the target gene under a stringent condition, the two oligonucleotides are subject to ligation in the presence of a suitable ligase. The ligation of the two oligonucleotides would indicate that the target DNA has a nucleotide variant at the locus being detected.

Detection of small genetic variations can also be accomplished by a variety of hybridization-based approaches. Allele-specific oligonucleotides are most useful. See Conner et al., Proc. Natl. Acad. Sci. USA, 80:278-282 (1983); Saiki et al, Proc. Natl. Acad. Sci. USA, 86:6230-6234 (1989). Oligonucleotide probes (allele-specific) hybridizing specifically to an gene allele having a particular gene variant at a particular locus but not to other alleles can be designed by methods known in the art. The probes can have a length of, e.g., from 10 to about 50 nucleotide bases. The target DNA and the oligonucleotide probe can be contacted with each other under conditions sufficiently stringent such that the nucleotide variant can be distinguished from the wild-type gene based on the presence or absence of hybridization. The probe can be labeled to provide detection signals. Alternatively, the allele-specific oligonucleotide probe can be used as a PCR amplification primer in an “allele-specific PCR” and the presence or absence of a PCR product of the expected length would indicate the presence or absence of a particular nucleotide variant.

Other useful hybridization-based techniques allow two single-stranded nucleic acids annealed together even in the presence of mismatch due to nucleotide substitution, insertion or deletion. The mismatch can then be detected using various techniques. For example, the annealed duplexes can be subject to electrophoresis. The mismatched duplexes can be detected based on their electrophoretic mobility that is different from the perfectly matched duplexes. See Cariello, Human Genetics, 42:726 (1988). Alternatively, in a RNase protection assay, a RNA probe can be prepared spanning the nucleotide variant site to be detected and having a detection marker. See Giunta et al., Diagn. Mol. Path., 5:265-270 (1996); Finkelstein et al., Genomics, 7:167-172 (1990); Kinszler et al., Science 251:1366-1370 (1991). The RNA probe can be hybridized to the target DNA or mRNA forming a heteroduplex that is then subject to the ribonuclease RNase A digestion. RNase A digests the RNA probe in the heteroduplex only at the site of mismatch. The digestion can be determined on a denaturing electrophoresis gel based on size variations. In addition, mismatches can also be detected by chemical cleavage methods known in the art.

In the mutS assay, a probe can be prepared matching the gene sequence surrounding the locus at which the presence or absence of a mutation is to be detected, except that a predetermined nucleotide is used at the variant locus. Upon annealing the probe to the target DNA to form a duplex, the E. coli mutS protein is contacted with the duplex. Since the mutS protein binds only to heteroduplex sequences containing a nucleotide mismatch, the binding of the mutS protein will be indicative of the presence of a mutation. See Modrich et al., Ann. Rev. Genet., 25:229-253 (1991).

A great variety of improvements and variations have been developed in the art on the basis of the above-described basic techniques, and can all be useful in detecting mutations or nucleotide variants in the present invention. For example, the “sunrise probes” or “molecular beacons” utilize the fluorescence resonance energy transfer (FRET) property and give rise to high sensitivity. See Wolf et al., Proc. Nat. Acad. Sci. USA, 85:8790-8794 (1988). Typically, a probe spanning the nucleotide locus to be detected are designed into a hairpinshaped structure and labeled with a quenching fluorophore at one end and a reporter fluorophore at the other end. In its natural state, the fluorescence from the reporter fluorophore is quenched by the quenching fluorophore due to the proximity of one fluorophore to the other. Upon hybridization of the probe to the target DNA, the 5′ end is separated apart from the 3′-end and thus fluorescence signal is regenerated. See Nazarenko et al., Nucleic Acids Res., 25:2516-2521 (1997); Rychlik et al., Nucleic Acids Res., 17:8543-8551 (1989); Sharkey et al., Bio/Technology 12:506-509 (1994); Tyagi et al., Nat. Biotechnol., 14:303-308 (1996); Tyagi et al., Nat. Biotechnol., 16:49-53 (1998). The homo-tag assisted non-dimer system (HANDS) can be used in combination with the molecular beacon methods to suppress primer-dimer accumulation. See Brownie et al., Nucleic Acids Res., 25:3235-3241 (1997).

Dye-labeled oligonucleotide ligation assay is a FRET-based method, which combines the OLA assay and PCR. See Chen et al., Genome Res. 8:549-556 (1998). TaqMan is another FRET-based method for detecting nucleotide variants. A TaqMan probe can be oligonucleotides designed to have the nucleotide sequence of the gene spanning the variant locus of interest and to differentially hybridize with different alleles. The two ends of the probe are labeled with a quenching fluorophore and a reporter fluorophore, respectively. The TaqMan probe is incorporated into a PCR reaction for the amplification of a target gene region containing the locus of interest using Taq polymerase. As Taq polymerase exhibits 5′-3′ exonuclease activity but has no 3′-5′ exonuclease activity, if the TaqMan probe is annealed to the target DNA template, the 5′-end of the TaqMan probe will be degraded by Taq polymerase during the PCR reaction thus separating the reporting fluorophore from the quenching fluorophore and releasing fluorescence signals. See Holland et al., Proc. Natl. Acad. Sci. USA, 88:7276-7280 (1991); Kalinina et al., Nucleic Acids Res., 25:1999-2004 (1997); Whitcombe et al., Clin. Chem., 44:918-923 (1998).

In addition, the detection in the present invention can also employ a chemiluminescence-based technique. For example, an oligonucleotide probe can be designed to hybridize to either the wild-type or a variant gene locus but not both. The probe is labeled with a highly chemiluminescent acridinium ester. Hydrolysis of the acridinium ester destroys chemiluminescence. The hybridization of the probe to the target DNA prevents the hydrolysis of the acridinium ester. Therefore, the presence or absence of a particular mutation in the target DNA is determined by measuring chemiluminescence changes. See Nelson et al., Nucleic Acids Res., 24:4998-5003 (1996).

The detection of genetic variation in the genes in accordance with the present invention can also be based on the “base excision sequence scanning” (BESS) technique. The BESS method is a PCR-based mutation scanning method. BESS T-Scan and BESS G-Tracker are generated which are analogous to T and G ladders of dideoxy sequencing. Mutations are detected by comparing the sequence of normal and mutant DNA. See, e.g., Hawkins et al., Electrophoresis, 20:1171-1176 (1999).

Another useful technique that is gaining increased popularity is mass spectrometry. See Graber et al., Curr. Opin. Biotechnol., 9:14-18 (1998). For example, in the primer oligo base extension (PROBE™) method, a target nucleic acid is immobilized to a solid-phase support. A primer is annealed to the target immediately 5′ upstream from the locus to be analyzed. Primer extension is carried out in the presence of a selected mixture of deoxyribonucleotides and dideoxyribonucleotides. The resulting mixture of newly extended primers is then analyzed by MALDI-TOF. See e.g., Monforte et al., Nat. Med., 3:360-362 (1997).

In addition, the microchip or microarray technologies are also applicable to the detection method of the present invention. Essentially, in microchips, a large number of different oligonucleotide probes are immobilized in an array on a substrate or carrier, e.g., a silicon chip or glass slide. Target nucleic acid sequences to be analyzed can be contacted with the immobilized oligonucleotide probes on the microchip. See Lipshutz et al., Biotechniques, 19:442-447 (1995); Chee et al., Science, 274:610-614 (1996); Kozal et al., Nat. Med. 2:753-759 (1996); Hacia et al., Nat. Genet., 14:441-447 (1996); Saiki et al., Proc. Natl. Acad. Sci. USA, 86:6230-6234 (1989); Gingeras et al., Genome Res., 8:435-448 (1998). Alternatively, the multiple target nucleic acid sequences to be studied are fixed onto a substrate and an array of probes is contacted with the immobilized target sequences. See Drmanac et al., Nat. Biotechnol., 16:54-58 (1998). Numerous microchip technologies have been developed incorporating one or more of the above described techniques for detecting mutations. The microchip technologies combined with computerized analysis tools allow fast screening in a large scale. The adaptation of the microchip technologies to the present invention will be apparent to a person of skill in the art apprised of the present disclosure. See, e.g., U.S. Pat. No. 5,925,525 to Fodor et al.

In a preferred embodiment, a biochip comprises any one or more of the nucleic acids, mutants, variants, fragments and corresponding peptides thereof identified in Tables 1 to 3.

In another preferred embodiment, a biochip comprises any one or more of antibodies and fragments thereof or aptamers specific for the gene products of nucleic acids, mutants, variants, fragments and corresponding peptides thereof identified in Tables 1 to 3. Initially, the molecular signature biomarker will be the base for the production of a customized microarray chip.

In another preferred embodiment, identification of novel therapeutic compositions as steroids and compositions for modulation of intraocular pressure comprises contacting the biochip comprising any one or more of the nucleic acids, mutants, variants, fragments, peptides thereof identified in Tables 1 to 3 with a library of compounds.

In another preferred embodiment, the biochip comprises antibodies specific for any one or more of the gene products of nucleic acids, mutants, variants, fragments and peptides thereof identified in Tables 1 to 3.

As is apparent from the above survey of the suitable detection techniques, it may or may not be necessary to amplify the target DNA, i.e., the gene, cDNA, mRNA, or a portion thereof to increase the number of target DNA molecule, depending on the detection techniques used. For example, most PCR-based techniques combine the amplification of a portion of the target and the detection of the mutations. PCR amplification is well known in the art and is disclosed in U.S. Pat. Nos. 4,683,195 and 4,800,159, both which are incorporated herein by reference. For non-PCR-based detection techniques, if necessary, the amplification can be achieved by, e.g., in vivo plasmid multiplication, or by purifying the target DNA from a large amount of tissue or cell samples. See generally, Sambrook et al., Molecular Cloning: A Laboratory Manual, 2.sup.nd ed., Cold Spring Harbor Laboratory, Cold Spring Harbor, N.Y., 1989. However, even with scarce samples, many sensitive techniques have been developed in which small genetic variations such as single-nucleotide substitutions can be detected without having to amplify the target DNA in the sample. For example, techniques have been developed that amplify the signal as opposed to the target DNA by, e.g., employing branched DNA or dendrimers that can hybridize to the target DNA. The branched or dendrimer DNAs provide multiple hybridization sites for hybridization probes to attach thereto thus amplifying the detection signals. See Detmer et al., J. Clin. Microbiol., 34:901-907 (1996); Nilsen et al., J. Theor. Biol., 187:273-284 (1997).

In yet another technique for detecting single nucleotide variations, the Invader™ assay utilizes a novel linear signal amplification technology that improves upon the long turnaround times required of the typical PCR DNA sequenced-based analysis. See Cooksey et al., Antimicrobial Agents and Chemotherapy 44:1296-1301 (2000). This assay is based on cleavage of a unique secondary structure formed between two overlapping oligonucleotides that hybridize to the target sequence of interest to form a “flap.” Each “flap” then generates thousands of signals per hour. Thus, the results of this technique can be easily read, and the methods do not require exponential amplification of the DNA target. The Invader™ system utilizes two short DNA probes, which are hybridized to a DNA target. The structure formed by the hybridization event is recognized by a special cleavase enzyme that cuts one of the probes to release a short DNA “flap.” Each released “flap” then binds to a fluorescently-labeled probe to form another cleavage structure. When the cleavase enzyme cuts the labeled probe, the probe emits a detectable fluorescence signal. See e.g. Lyamichev et al., Nat. Biotechnol., 17:292-296 (1999).

The rolling circle method is another method that avoids exponential amplification. Lizardi et al., Nature Genetics, 19:225-232 (1998) (which is incorporated herein by reference). For example, SNIPER™, a commercial embodiment of this method, is a sensitive, high-throughput SNP scoring system designed for the accurate fluorescent detection of specific variants. For each nucleotide variant, two linear, allele-specific probes are designed. The two allele-specific probes are identical with the exception of the 3′-base, which is varied to complement the variant site. In the first stage of the assay, target DNA is denatured and then hybridized with a pair of single, allele-specific, open-circle oligonucleotide probes. When the 3′-base exactly complements the target DNA, ligation of the probe will preferentially occur. Subsequent detection of the circularized oligonucleotide probes is by rolling circle amplification, whereupon the amplified probe products are detected by fluorescence. See Clark and Pickering, Life Science News 6, 2000, Amersham Pharmacia Biotech (2000).

A number of other techniques that avoid amplification all together include, e.g., surface-enhanced resonance Raman scattering (SERRS), fluorescence correlation spectroscopy, and single-molecule electrophoresis. In addition, the allele-specific oligonucleotides (ASO) can also be used in in situ hybridization using tissues or cells as samples. The oligonucleotide probes which can hybridize differentially with the wild-type gene sequence or the gene sequence harboring a mutation may be labeled with radioactive isotopes, fluorescence, or other detectable markers. In situ hybridization techniques are well known in the art and their adaptation to the present invention for detecting the presence or absence of a nucleotide variant in the one or more gene of a particular individual should be apparent to a skilled artisan apprised of this disclosure.

Protein-based detection techniques may also prove to be useful, especially when the nucleotide variant causes amino acid substitutions or deletions or insertions or frameshift that affect the protein primary, secondary or tertiary structure. To detect the amino acid variations, protein sequencing techniques may be used. For example, a protein or fragment thereof corresponding to an gene can be synthesized by recombinant expression using a DNA fragment isolated from an individual to be tested. Preferably, a cDNA fragment of no more than 100 to 150 base pairs encompassing the polymorphic locus to be determined is used. The amino acid sequence of the peptide can then be determined by conventional protein sequencing methods. Alternatively, the HPLC-microscopy tandem mass spectrometry technique can be used for determining the amino acid sequence variations. In this technique, proteolytic digestion is performed on a protein, and the resulting peptide mixture is separated by reversed-phase chromatographic separation. Tandem mass spectrometry is then performed and the data collected therefrom is analyzed. See Gatlin et al., Anal. Chem., 72:757-763 (2000).

Other useful protein-based detection techniques include immunoaffinity assays based on antibodies selectively immunoreactive with mutant gene encoded protein according to the present invention. The method for producing such antibodies is known in the art. Antibodies can be used to immunoprecipitate specific proteins from solution samples or to immunoblot proteins separated by, e.g., polyacrylamide gels. Immunocytochemical methods can also be used in detecting specific protein polymorphisms in tissues or cells. Other well-known antibody-based techniques can also be used including, e.g., enzyme-linked immunosorbent assay (ELISA), radioimmunoassay (RIA), immunoradiometric assays (IRMA) and immunoenzymatic assays (IEMA), including sandwich assays using monoclonal or polyclonal antibodies. See e.g., U.S. Pat. Nos. 4,376,110 and 4,486,530, both of which are incorporated herein by reference.

Accordingly, the presence or absence of one or more genes nucleotide variant or amino acid variant in an individual can be determined using any of the detection methods described above.

Typically, once the presence or absence of one or more gene nucleotide variants or amino acid variants is determined (or the status of the biomarkers in Tables 1 to 3), physicians or genetic counselors or patients or other researchers may be informed of the result. Specifically the result can be cast in a transmittable form that can be communicated or transmitted to other researchers or physicians or genetic counselors or patients. Such a form can vary and can be tangible or intangible. The result with regard to the presence or absence of a nucleotide variant of the present invention in the individual tested can be embodied in descriptive statements, diagrams, photographs, charts, images or any other visual forms. For example, images of gel electrophoresis of PCR products can be used in explaining the results. Diagrams showing where a variant occurs in an individual's gene are also useful in indicating the testing results. The statements and visual forms can be recorded on a tangible media such as papers, computer readable media such as floppy disks, compact disks, etc., or on an intangible media, e.g., an electronic media in the form of email or website on internet or intranet. In addition, the result with regard to the presence or absence of a nucleotide variant or amino acid variant in the individual tested can also be recorded in a sound form and transmitted through any suitable media, e.g., analog or digital cable lines, fiber optic cables, etc., via telephone, facsimile, wireless mobile phone, internet phone and the like.

Thus, the information and data on a test result can be produced anywhere in the world and transmitted to a different location. For example, when a genotyping assay is conducted offshore, the information and data on a test result may be generated and cast in a transmittable form as described above. The test result in a transmittable form thus can be imported into the U.S. Accordingly, the present invention also encompasses a method for producing a transmittable form of information on the genotype of the two or more suspected cancer samples from an individual. The method comprises the steps of (1) determining the genotype of the DNA from the samples according to methods of the present invention; and (2) embodying the result of the determining step in a transmittable form. The transmittable form is the product of the production method.

### Candidate Therapeutic Agents

In a preferred embodiment, a method of identifying candidate therapeutic agents for treatment of diseases such as glaucoma or other diseases where steroids would be effective, comprising: (a) contacting a biological sample from a steroid non-responder and steroid responder patient, with the candidate agent and determining the level of expression of one or more biomarkers or identification of a biomarker profile as compared between the steroid responder versus the non-responder; (b) determining the level of expression of a corresponding biomarker or biomarkers in an aliquot of the biological sample not contacted with the candidate agent; (c) observing the effect of the candidate agent by comparing the level of expression of the biomarker or biomarkers in the aliquot of the biological sample contacted with the candidate agent and the level of expression of the corresponding biomarker or biomarkers in the aliquot of the biological sample not contacted with the candidate agent; and (d) identifying said agent from said observed effect, wherein an at least 10% difference between the level of expression of the biomarker gene or combination of biomarker genes (biomarker profile) in the aliquot of the biological sample contacted with the candidate agent and the level of expression of the corresponding biomarker gene or combination of biomarker genes in the aliquot of the biological sample not contacted with the candidate agent is an indication of an effect of the candidate agent.

In preferred embodiments, the effects of the drug are correlated with the expression of certain biomarkers or the biomarker profiles and identify those individuals who will be a responder or whether certain individuals would be at risk of developing, for example, intraocular pressure as a result of steroid treatment. The drugs identified would thus be used as opposed to steroid treatments.

In another embodiment of the invention, a pharmaceutical preparation comprising an agent according to the invention is provided.

In another preferred embodiment of the invention, a method of producing a drug comprising the steps of the method according to the invention (i) synthesizing the candidate agent identified in step (c) above or an analog or derivative thereof in an amount sufficient to provide said drug in a therapeutically effective amount to a subject; and/or (ii) combining the drug candidate the candidate agent identified in step (c) above or an analog or derivative thereof with a pharmaceutically acceptable carrier.

In some embodiments it is desirable to express the biomolecules that comprise a biomarker, in a vector and in cells. The applications of such combinations are unlimited. The vectors and cells expressing the one or more biomolecules can be used in assays, kits, drug discovery, diagnostics, prognostics and the like. The cells can be stem cells isolated from the bone marrow as a progenitor cell, or cells obtained from any other source, such as for example, ATCC.

In another preferred embodiment, an agent or drug is identified by methods comprising culturing an isolated cell wherein a cellular receptor has been regulated using the methods of the invention, for example, regulation (i.e., up-regulation, or inhibition of expression of a receptor) and, administering a candidate therapeutic agent to the cultured cell; correlating expression levels and phosphorylation of the receptor in the presence or absence of a candidate therapeutic agent as compared to a normal cell and a cell with a regulated receptor, cultured in the presence of a candidate therapeutic agent, wherein a drug is identified based on desirable therapeutic outcomes. For example, a drug which increases expression of a receptor, decreases expression of a receptor, phosphorylates or de-phosphorylates a receptor, responses to steroids and the like, thereby, identifying candidate therapeutic agents that regulate receptors.

Another suitable method for prognosis, risk assessment, and candidate drug discovery includes contacting a test sample with a cell expressing a receptor or gene thereof, an allele or fragment thereof; and detecting interaction of the test sample with the gene, an allele or fragment thereof, or expression product of the gene, an allele or fragment thereof. The desired gene, an allele or fragment thereof, or expression product of the gene, an allele or fragment thereof suitably can be detectably labeled e.g. with a fluorescent or radioactive component.

In another preferred embodiment, a cell from a patient is isolated and contacted with a candidate therapeutic molecule. The genes, expression products thereof, are monitored to identify which genes or expression products are regulated by the drug. Interference RNA's can then be synthesized to regulate the identified genes, expression products that are regulated by the drug and thus, provide therapeutic oligonucleotides. These can be tailored to individual patients, which is advantageous as different patients do not effectively respond to the same drugs equally. Thus, the oligonucleotides would provide a cheaper and individualized treatment than conventional drug treatments.

Probes may also be used for the detection of related sequences, and should preferably have at least 50% sequence identity or homology to any of the identified genes encoding sequences, more preferably at least about 60, 70, 75, 80, 85, 90 or 95 percent sequence identity to any of the identified gene encoding sequences (sequence identity determinations discussed above, including use of BLAST program). The hybridization probes of the subject invention may be DNA or RNA and may be derived from the sequences of the invention or from genomic sequences including promoters, enhancers, and introns of the gene.

The polynucleotide sequences encoding a target gene may be used in Southern or Northern analysis, dot blot, or other membrane-based technologies; in PCR technologies; in dipstick, pin, and multiformat ELISA-like assays; and in microarrays utilizing fluids or tissues from patients to detect altered target gene expression. Gel-based mobility-shift analyses may be employed. Other suitable qualitative or quantitative methods are well known in the art.

Identity of genes, or variants thereof, can be verified using techniques well known in the art and have been described above. Briefly, examples include but are not limited to, nucleic acid sequencing of amplified genes, hybridization techniques such as single nucleic acid polymorphism analysis (SNP), microarrays wherein the molecule of interest is immobilized on a biochip. Overlapping cDNA clones can be sequenced by the dideoxy chain reaction using fluorescent dye terminators and an ABI sequencer (Applied Biosystems, Foster City, Calif.). Any type of assay wherein one component is immobilized may be carried out using the substrate platforms of the invention. Bioassays utilizing an immobilized component are well known in the art. Examples of assays utilizing an immobilized component include for example, immunoassays, analysis of protein-protein interactions, analysis of protein-nucleic acid interactions, analysis of nucleic acid-nucleic acid interactions, receptor binding assays, enzyme assays, phosphorylation assays, diagnostic assays for determination of disease state, genetic profiling for drug compatibility analysis, SNP detection, etc.

Identification of a nucleic acid sequence capable of binding to a biomolecule of interest can be achieved by immobilizing a library of nucleic acids onto the substrate surface so that each unique nucleic acid was located at a defined position to form an array. The array would then be exposed to the biomolecule under conditions which favored binding of the biomolecule to the nucleic acids. Non-specifically binding biomolecules could be washed away using mild to stringent buffer conditions depending on the level of specificity of binding desired. The nucleic acid array would then be analyzed to determine which nucleic acid sequences bound to the biomolecule. Preferably the biomolecules would carry a fluorescent tag for use in detection of the location of the bound nucleic acids.

An assay using an immobilized array of nucleic acid sequences may be used for determining the sequence of an unknown nucleic acid; single nucleotide polymorphism (SNP) analysis; analysis of gene expression patterns from a particular species, tissue, cell type, etc.; gene identification; etc.

Additional diagnostic uses for oligonucleotides designed from the sequences encoding a desired gene expression product may involve the use of PCR. These oligomers may be chemically synthesized, generated enzymatically, or produced in vitro. Oligomers will preferably contain a fragment of a polynucleotide encoding the expression products, or a fragment of a polynucleotide complementary to the polynucleotides, and will be employed under optimized conditions for identification of a specific gene. Oligomers may also be employed under less stringent conditions for detection or quantitation of closely-related DNA or RNA sequences.

In further embodiments, oligonucleotides or longer fragments derived from any of the polynucleotide sequences, may be used as targets in a microarray. The microarray can be used to monitor the identity and/or expression level of large numbers of genes and gene transcripts simultaneously to identify genes with which target genes or its product interacts and/or to assess the efficacy of candidate therapeutic agents in regulating expression products of genes that mediate, for example, neurological disorders. This information may be used to determine gene function, and to develop and monitor the activities of therapeutic agents.

Candidate agents include numerous chemical classes, though typically they are organic compounds including small organic compounds, nucleic acids including oligonucleotides, and peptides. Small organic compounds suitably may have e.g. a molecular weight of more than about 40 or 50 yet less than about 2,500. Candidate agents may comprise functional chemical groups that interact with proteins and/or DNA.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Therapeutic agent assays of the invention suitably include, animal models, cell-based systems and non-cell based systems. Preferably, identified genes, variants, fragments, or oligopeptides thereof are used for identifying agents of therapeutic interest, e.g. by screening libraries of compounds or otherwise identifying compounds of interest by any of a variety of drug screening or analysis techniques. The gene, allele, fragment, or oligopeptide thereof employed in such screening may be free in solution, affixed to a solid support, borne on a cell surface, or located intracellularly.

Another technique for drug screening provides for high throughput screening of compounds having suitable binding affinity to the protein of interest (see, e.g., Geysen et al., 1984, PCT application WO84/03564). In this method, large numbers of different small test compounds are synthesized on a solid substrate. The test compounds are reacted with identified genes, or fragments thereof, and washed. Bound molecules are then detected by methods well known in the art. Alternatively, non-neutralizing antibodies can be used to capture the peptide and immobilize it on a solid support.

The methods of screening of the invention comprise using screening assays to identify, from a library of diverse molecules, one or more compounds having a desired activity. A “screening assay” is a selective assay designed to identify, isolate, and/or determine the structure of, compounds within a collection that have a preselected activity. By “identifying” it is meant that a compound having a desirable activity is isolated, its chemical structure is determined (including without limitation determining the nucleotide and amino acid sequences of nucleic acids and polypeptides, respectively) the structure of and, additionally or alternatively, purifying compounds having the screened activity). Biochemical and biological assays are designed to test for activity in a broad range of systems ranging from protein-protein interactions, enzyme catalysis, small molecule-protein binding, to cellular functions. Such assays include automated, semi-automated assays and HTS (high throughput screening) assays.

In HTS methods, many discrete compounds are preferably tested in parallel by robotic, automatic or semi-automatic methods so that large numbers of test compounds are screened for a desired activity simultaneously or nearly simultaneously. It is possible to assay and screen up to about 6,000 to 20,000, and even up to about 100,000 to 1,000,000 different compounds a day using the integrated systems of the invention.

Typically in HTS, target molecules are administered or cultured with isolated cells with modulated receptors, including the appropriate controls.

In one embodiment, screening comprises contacting each cell culture with a diverse library of member compounds, some of which are ligands of the target, under conditions where complexes between the target and ligands can form, and identifying which members of the libraries are present in such complexes. In another non limiting modality, screening comprises contacting a target with a diverse library of member compounds, some of which are inhibitors (or activators) of the target, under conditions where a product or a reactant of the reaction catalyzed by the enzyme produce a detectable signal. In the latter modality, inhibitors of target decrease the signal from a detectable product or increase a signal from a detectable reactant (or vice-versa for activators).

Chemical Libraries:

Developments in combinatorial chemistry allow the rapid and economical synthesis of hundreds to thousands of discrete compounds. These compounds are typically arrayed in moderate-sized libraries of small molecules designed for efficient screening. Combinatorial methods, can be used to generate unbiased libraries suitable for the identification of novel compounds. In addition, smaller, less diverse libraries can be generated that are descended from a single parent compound with a previously determined biological activity. In either case, the lack of efficient screening systems to specifically target therapeutically relevant biological molecules produced by combinational chemistry such as inhibitors of important enzymes hampers the optimal use of these resources.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol. Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 90:6909-6913 (1993)); vinylogous polypeptides (Hagihara, et al., J. Amer. Chem. Soc. 114:6568 (1992)); nonpeptidal peptidomimetics with .beta.-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

High throughput screening can be used to measure the effects of drugs on complex molecular events such as signal transduction pathways, as well as cell functions including, but not limited to, cell function, apoptosis, cell division, cell adhesion, locomotion, exocytosis, and cell-cell communication. Multicolor fluorescence permits multiple targets and cell processes to be assayed in a single screen. Cross-correlation of cellular responses will yield a wealth of information required for target validation and lead optimization.

In another aspect, the present invention provides a method for analyzing cells comprising providing an array of locations which contain multiple cells wherein the cells contain one or more fluorescent reporter molecules; scanning multiple cells in each of the locations containing cells to obtain fluorescent signals from the fluorescent reporter molecule in the cells; converting the fluorescent signals into digital data; and utilizing the digital data to determine the distribution, environment or activity of the fluorescent reporter molecule within the cells.

A major component of the new drug discovery paradigm is a continually growing family of fluorescent and luminescent reagents that are used to measure the temporal and spatial distribution, content, and activity of intracellular ions, metabolites, macromolecules, and organelles. Classes of these reagents include labeling reagents that measure the distribution and amount of molecules in living and fixed cells, environmental indicators to report signal transduction events in time and space, and fluorescent protein biosensors to measure target molecular activities within living cells. A multiparameter approach that combines several reagents in a single cell is a powerful new tool for drug discovery.

This method relies on the high affinity of fluorescent or luminescent molecules for specific cellular components. The affinity for specific components is governed by physical forces such as ionic interactions, covalent bonding (which includes chimeric fusion with protein-based chromophores, fluorophores, and lumiphores), as well as hydrophobic interactions, electrical potential, and, in some cases, simple entrapment within a cellular component. The luminescent probes can be small molecules, labeled macromolecules, or genetically engineered proteins, including, but not limited to green fluorescent protein chimeras.

Those skilled in this art will recognize a wide variety of fluorescent reporter molecules that can be used in the present invention, including, but not limited to, fluorescently labeled biomolecules such as proteins, phospholipids, RNA and DNA hybridizing probes. Similarly, fluorescent reagents specifically synthesized with particular chemical properties of binding or association have been used as fluorescent reporter molecules (Barak et al., (1997), J. Biol. Chem. 272:27497-27500; Southwick et al., (1990), Cytometry 11:418-430; Tsien (1989) in Methods in Cell Biology, Vol. 29 Taylor and Wang (eds.), pp. 127-156). Fluorescently labeled antibodies are particularly useful reporter molecules due to their high degree of specificity for attaching to a single molecular target in a mixture of molecules as complex as a cell or tissue.

The luminescent probes can be synthesized within the living cell or can be transported into the cell via several non-mechanical modes including diffusion, facilitated or active transport, signal-sequence-mediated transport, and endocytotic or pinocytotic uptake. Mechanical bulk loading methods, which are well known in the art, can also be used to load luminescent probes into living cells (Barber et al. (1996), Neuroscience Letters 207:17-20; Bright et al. (1996), Cytometry 24:226-233; McNeil (1989) in Methods in Cell Biology, Vol. 29, Taylor and Wang (eds.), pp. 153-173). These methods include electroporation and other mechanical methods such as scrape-loading, bead-loading, impact-loading, syringe-loading, hypertonic and hypotonic loading. Additionally, cells can be genetically engineered to express reporter molecules, such as GFP, coupled to a protein of interest as previously described (Chalfie and Prasher U.S. Pat. No. 5,491,084; Cubitt et al. (1995), Trends in Biochemical Science 20:448-455).

Once in the cell, the luminescent probes accumulate at their target domain as a result of specific and high affinity interactions with the target domain or other modes of molecular targeting such as signal-sequence-mediated transport. Fluorescently labeled reporter molecules are useful for determining the location, amount and chemical environment of the reporter. For example, whether the reporter is in a lipophilic membrane environment or in a more aqueous environment can be determined (Giuliano et al. (1995), Ann. Rev. of Biophysics and Biomolecular Structure 24:405-434; Giuliano and Taylor (1995), Methods in Neuroscience 27.1-16). The pH environment of the reporter can be determined (Bright et al. (1989), J. Cell Biology 104:1019-1033; Giuliano et al. (1987), Anal. Biochem. 167:362-371; Thomas et al. (1979), Biochemistry 18:2210-2218). It can be determined whether a reporter having a chelating group is bound to an ion, such as Ca⁺⁺, or not (Bright et al. (1989), In Methods in Cell Biology, Vol. 30, Taylor and Wang (eds.), pp. 157-192; Shimoura et al. (1988), J. of Biochemistry (Tokyo) 251:405-410; Tsien (1989) In Methods in Cell Biology, Vol. 30, Taylor and Wang (eds.), pp. 127-156).

Furthermore, certain cell types within an organism may contain components that can be specifically labeled that may not occur in other cell types. For example, neural cells often contain polarized membrane components. That is, these cells asymmetrically distribute macromolecules along their plasma membrane. Connective or supporting tissue cells often contain granules in which are trapped molecules specific to that cell type (e.g., heparin, histamine, serotonin, etc.). Most muscular tissue cells contain a sarcoplasmic reticulum, a specialized organelle whose function is to regulate the concentration of calcium ions within the cell cytoplasm. Many nervous tissue cells contain secretory granules and vesicles in which are trapped neurohormones or neurotransmitters. Therefore, fluorescent molecules can be designed to label not only specific components within specific cells, but also specific cells within a population of mixed cell types.

Those skilled in the art will recognize a wide variety of ways to measure fluorescence. For example, some fluorescent reporter molecules exhibit a change in excitation or emission spectra, some exhibit resonance energy transfer where one fluorescent reporter loses fluorescence, while a second gains in fluorescence, some exhibit a loss (quenching) or appearance of fluorescence, while some report rotational movements (Giuliano et al. (1995), Ann. Rev. of Biophysics and Biomol. Structure 24:405-434; Giuliano et al. (1995), Methods in Neuroscience 27:1-16).

The whole procedure can be fully automated. For example, sampling of sample materials may be accomplished with a plurality of steps, which include withdrawing a sample from a sample container and delivering at least a portion of the withdrawn sample to test cell culture (e.g., a cell culture wherein gene expression is regulated). Sampling may also include additional steps, particularly and preferably, sample preparation steps. In one approach, only one sample is withdrawn into the auto-sampler probe at a time and only one sample resides in the probe at one time. In other embodiments, multiple samples may be drawn into the auto-sampler probe separated by solvents. In still other embodiments, multiple probes may be used in parallel for auto sampling.

In the general case, sampling can be effected manually, in a semi-automatic manner or in an automatic manner. A sample can be withdrawn from a sample container manually, for example, with a pipette or with a syringe-type manual probe, and then manually delivered to a loading port or an injection port of a characterization system. In a semi-automatic protocol, some aspect of the protocol is effected automatically (e.g., delivery), but some other aspect requires manual intervention (e.g., withdrawal of samples from a process control line). Preferably, however, the sample(s) are withdrawn from a sample container and delivered to the characterization system, in a fully automated manner—for example, with an auto-sampler.

In one embodiment, auto-sampling may be done using a microprocessor controlling an automated system (e.g., a robot arm). Preferably, the microprocessor is user-programmable to accommodate libraries of samples having varying arrangements of samples (e.g., square arrays with “n-rows” by “n-columns,” rectangular arrays with “n-rows” by “m-columns,” round arrays, triangular arrays with “r-” by “r-” by “r-” equilateral sides, triangular arrays with “r-base” by “s-” by “s-” isosceles sides, etc., where n, m, r, and s are integers).

Automated sampling of sample materials optionally may be effected with an auto-sampler having a heated injection probe (tip). An example of one such auto sampler is disclosed in U.S. Pat. No. 6,175,409 B1 (incorporated by reference).

According to the present invention, one or more systems, methods or both are used to identify a plurality of sample materials. Though manual or semi-automated systems and methods are possible, preferably an automated system or method is employed. A variety of robotic or automatic systems are available for automatically or programmably providing predetermined motions for handling, contacting, dispensing, or otherwise manipulating materials in solid, fluid liquid or gas form according to a predetermined protocol. Such systems may be adapted or augmented to include a variety of hardware, software or both to assist the systems in determining mechanical properties of materials. Hardware and software for augmenting the robotic systems may include, but are not limited to, sensors, transducers, data acquisition and manipulation hardware, data acquisition and manipulation software and the like. Exemplary robotic systems are commercially available from CAVRO Scientific Instruments (e.g., Model NO. RSP9652) or BioDot (Microdrop Model 3000).

Generally, the automated system includes a suitable protocol design and execution software that can be programmed with information such as synthesis, composition, location information or other information related to a library of materials positioned with respect to a substrate. The protocol design and execution software is typically in communication with robot control software for controlling a robot or other automated apparatus or system. The protocol design and execution software is also in communication with data acquisition hardware/software for collecting data from response measuring hardware. Once the data is collected in the database, analytical software may be used to analyze the data, and more specifically, to determine properties of the candidate drugs, or the data may be analyzed manually.

In another preferred embodiment, the assaying of the candidate drugs or samples with the cell culture is combined with one or more methods. In one embodiment, a sample can be pre-fractionated according to size of proteins in a sample using size exclusion chromatography. For a biological sample wherein the amount of sample available is small, preferably a size selection spin column is used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction can then be analyzed by immunoassays, gas phase ion spectrometry, and the like, for the detection of compounds.

In another embodiment, a sample can be pre-fractionated by anion exchange chromatography. Anion exchange chromatography allows pre-fractionation of the proteins in a sample roughly according to their charge characteristics. For example, a Q anion-exchange resin can be used (e.g., Q HyperD F, Biosepra), and a sample can be sequentially eluted with eluants having different pH's. Anion exchange chromatography allows separation of compounds in a sample that are more negatively charged from other types of compounds. Proteins that are eluted with an eluant having a high pH is likely to be weakly negatively charged, and a fraction that is eluted with an eluant having a low pH is likely to be strongly negatively charged. Thus, in addition to reducing complexity of a sample, anion exchange chromatography separates proteins according to their binding characteristics.

In yet another embodiment, a sample can be pre-fractionated by heparin chromatography. Heparin chromatography allows pre-fractionation of the compounds in a sample also on the basis of affinity interaction with heparin and charge characteristics. Heparin, a sulfated mucopolysaccharide, will bind compounds with positively charged moieties and a sample can be sequentially eluted with eluants having different pH's or salt concentrations. Samples eluted with an eluant having a low pH are more likely to be weakly positively charged. Samples eluted with an eluant having a high pH are more likely to be strongly positively charged. Thus, heparin chromatography also reduces the complexity of a sample and separates samples according to their binding characteristics.

In yet another embodiment, a sample can be pre-fractionated by isolating proteins that have a specific characteristic, e.g. are glycosylated. For example, a CSF sample can be fractionated by passing the sample over a lectin chromatography column (which has a high affinity for sugars). Glycosylated proteins will bind to the lectin column and non-glycosylated proteins will pass through the flow through. Glycosylated proteins are then eluted from the lectin column with an eluant containing a sugar, e.g., N-acetyl-glucosamine and are available for further analysis.

Thus there are many ways to reduce the complexity of a sample based on the binding properties of the proteins in the sample, or the characteristics of the proteins in the sample.

### Kits

The present invention also provides a kit for genotyping the one or more genes, i.e., determining the presence or absence of one or more of the nucleotide or amino acid variants in one or more genes in a sample obtained from a patient. The kit may include a carrier for the various components of the kit. The carrier can be a container or support, in the form of, e.g., bag, box, tube, rack, and is optionally compartmentalized. The carrier may define an enclosed confinement for safety purposes during shipment and storage. The kit also includes various components useful in detecting nucleotide or amino acid variants discovered in accordance with the present invention using the above-discussed detection techniques.

In another preferred embodiment, a kit for predicting risk for steroid responses comprises one or more of the following nucleic acids, variants, mutants and fragments thereof: NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6VOD2, ATPase, fr transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4, variants, mutants, homologs, alleles and fragments, or sequences complementary thereto.

The kits of the invention can include the probes and reagents described above for detecting the one or more biomarkers of the invention, and optionally include reagents and probes for analyzing one or more genes, or for re-analysis of one or more of the biomarkers of the invention.

In one embodiment, the detection kit includes one or more oligonucleotides useful in detecting one or more of the nucleotide variants in one or more genes. Preferably, the oligonucleotides are allele-specific, i.e., are designed such that they hybridize only to a mutant gene containing a particular nucleotide variant discovered in accordance with the present invention, under stringent conditions. Thus, the oligonucleotides can be used in mutation-detecting techniques such as allele-specific oligonucleotides (ASO), allele-specific PCR, TAQMAN, chemiluminescence-based techniques, molecular beacons, and improvements or derivatives thereof, e.g., microchip technologies. The oligonucleotides in this embodiment preferably have a nucleotide sequence that matches a nucleotide sequence of a variant gene allele containing a nucleotide variant to be detected. The length of the oligonucleotides in accordance with this embodiment of the invention can vary depending on its nucleotide sequence and the hybridization conditions employed in the detection procedure. Preferably, the oligonucleotides contain from about 10 nucleotides to about 100 nucleotides, more preferably from about 15 to about 75 nucleotides, e.g., contiguous span of 18, 19, 20, 21, 22, 23, 24 or 25 to 21, 22, 23, 24, 26, 27, 28, 29 or 30 nucleotide residues of a an gene nucleic acid. Under most conditions, a length of 18 to 30 may be optimum. In any event, the oligonucleotides should be designed such that it can be used in distinguishing one nucleotide variant from another at a particular locus under predetermined stringent hybridization conditions. A nucleotide variant can be located anywhere on the gene or it can be located at the center or within one (1) nucleotide of the center of the oligonucleotides, or at the 3′ or 5′ end of the oligonucleotides. The hybridization of an oligonucleotide with a nucleic acid and the optimization of the length and hybridization conditions should be apparent to a person of skill in the art. See generally, Sambrook et al., Molecular Cloning: A Laboratory Manual, 2^{nd} ed., Cold Spring Harbor Laboratory, Cold Spring Harbor, N.Y., 1989. Notably, the oligonucleotides in accordance with this embodiment are also useful in mismatch-based detection techniques described above, such as electrophoretic mobility shift assay, RNase protection assay, mutS assay, etc.

In another embodiment of this invention, the kit includes one or more oligonucleotides suitable for use in detecting techniques such as ARMS, oligonucleotide ligation assay (OLA), and the like. The oligonucleotides in this embodiment include a gene sequence of about 10 to about 100 nucleotides, preferably from about 15 to about 75 nucleotides, e.g., contiguous span of 18, 19, 20, 21, 22, 23, 24 or 25 to 21, 22, 23, 24, 26, 27, 28, 29 or 30 nucleotide residues immediately 5′ upstream from the nucleotide variant to be analyzed. The 3′ end nucleotide in such oligonucleotides is a nucleotide variant in accordance with this invention.

The oligonucleotides in the detection kit can be labeled with any suitable detection marker including but not limited to, radioactive isotopes, fluorophores, biotin, enzymes (e.g., alkaline phosphatase), enzyme substrates, ligands and antibodies, etc. See Jablonski et al., Nucleic Acids Res., 14:6115-6128 (1986); Nguyen et al., Biotechniques, 13:116-123 (1992); Rigby et al., J. Mol. Biol., 113:237-251 (1977). Alternatively, the oligonucleotides included in the kit are not labeled, and instead, one or more markers are provided in the kit so that users may label the oligonucleotides at the time of use.

In another embodiment of the invention, the detection kit contains one or more antibodies selectively immunoreactive with certain proteins or polypeptides (encoded by the genes) containing specific amino acid variants discovered in the present invention.

Various other components useful in the detection techniques may also be included in the detection kit of this invention. Examples of such components include, but are not limited to, Taq polymerase, deoxyribonucleotides, dideoxyribonucleotides other primers suitable for the amplification of a target DNA sequence, RNase A, mutS protein, and the like. In addition, the detection kit preferably includes instructions on using the kit for detecting nucleotide variants in gene sequences, or other nucleic acid molecules, e.g. RNA.

### Therapeutic Agents

In some aspects, the methods, biomarkers, and compositions of the invention are useful for selecting a therapeutic treatment for a patient having a particular biomarker profile. According to these embodiments, the set of biomarkers is used to select a treatment for a steroid non-responder, or a patient wherein a steroid would cause adverse or undesired effects such an increase in intraocular pressure, based on the association of a biomarker signature with response or lack of response to a particular therapeutic or class of therapeutics. In one aspect of the invention, the methods and biomarkers are used to classify patients as responders and non-responders to a particular therapeutic.

The compounds described herein can be incorporated into pharmaceutical compositions. Such compositions typically include the active ingredient and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from pressured container or dispenser which contains a suitable propellant, e.g., a gas such as carbon dioxide, or a nebulizer. Such methods include those described in U.S. Pat. No. 6,468,798. Compositions for inhalation can also include propellants, surfactants, and other additives, e.g., to improve dispersion, flow, and bioavailability.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Embodiments of inventive compositions and methods are illustrated in the following examples. These examples are provided for illustrative purposes and are not considered limitations on the scope of inventive compositions and methods.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

### Materials and Methods

This study was compliant with the Declaration of Helsinki, the institutional review board of the University of Miami, and the Health Insurance Portability and Accountability Act. Informed consent was obtained from the subjects after explanation of the nature and possible consequences of the study.

Patients who were recruited were offered treatment with IVTA for various retinal diseases or who had received IVTA in the past. Each patient was treated with IVTA, 4 mg in 0.1 cc, for a variety of indications, at the discretion of the treating physician. Inclusion criteria included age greater than 18 years and the ability to give appropriate informed consent. Exclusion criteria included prior pars plana vitrectomy in the study eye, anterior-segment neovascularization in the study eye, the use of any medication intended to lower IOP in the study eye, or a history of glaucoma, suspected glaucoma, or ocular hypertension in the study eye.

All IOP measurements were as per the treating physician's standard practice, typically either applanation tonometry or Tono-pen (Reichert, Depew, N.Y.). No standardized IOP measurement protocol was followed. Intraocular pressure (TOP) was recorded in the study eye prior to treatment with IVTA (baseline IOP). Follow-up was performed at the discretion of the treating physician. On each subsequent visit, IOP was recorded (follow-up IOP) for up to 1 year, or until the study eye was treated with any subsequent intraocular surgery, any subsequent intravitreal injection, or any medication intended to lower IOP. Therefore, during the time period relevant to this study, each eye received only one intravitreal injection.

All treatment decisions were at the discretion of the treating physician. The highest follow-up IOP was recorded (maximum IOP). The primary outcome measure was the difference between maximum IOP and baseline IOP was calculated (A IOP), with a positive value indicating a rise in IOP.

Peripheral venous blood was obtained for DNA analysis. Genomic DNA was extracted from peripheral blood leukocytes using Gentra PUREGENE Cell Kit (Qiagen, Inc., Valencia, Calif.). Allelic status of the glucocorticoid receptor polymorphisms was determined by standard polymerase chain reaction and direct sequencing. Restriction fragment length polymorphism analysis was used for the BclI polymorphism. Some DNA samples were genotyped by single-base extension reactions discriminated by matrix-assisted laser desorption ionization, time-of-flight mass spectrometry (Sequenom, Inc., San Diego, Calif.).

The single-base extension reaction performed by the Sequenom genotyping platform is a two step process. First, the region containing the SNP is amplified. Then, a primer ending at the polymorphic site is used for the single-base extension reaction. The products are then sorted by matrix-assisted laser desorption ionization, time-of-flight mass spectrometry (MALDI-TOF MS).

Briefly, primers for PCR and single base extension reactions were designed by using the MassARRAY Assay Design 3.0 software package (Sequenom, Inc., San Diego, Calif.). One milliliter of 2.5-10 ng/μL genomic DNA was combined with 1.85 μL of water, 0.1 μL of 25 mM dNTPs (Invitrogen Corp., Carlsbad, Calif.), 0.1 μL of 5 units/μL HOTSTAR Taq (Qiagen Inc., Valencia Calif.), 0.625 μL of 10× HotStar PCR buffer containing 15 mM MgCl₂, 1 μL PCR primers mixed together at a concentration of 500 nM for multiplexed reactions, and 0.325 μL of 25 mM MgCl₂. Reactions were heated at 95° C. for 15 min followed 1 by 45 cycles at 95° C. for 20 seconds, 56° C. for 30 seconds, and 72° C. for 1 minute and a final incubation at 72° C. for 3 minutes. After PCR amplification, remaining dNTPs were dephosphorylated by adding 1.5 μL of water, 0.17 μL of 10×SAP buffer (Sequenom, Inc., San Diego, Calif.), and 0.3 units of shrimp alkaline phosphatase (Sequenom, Inc., San Diego, Calif.). The reaction was placed at 37° C. for 20 minutes, and the enzyme was deactivated by incubating at 85° C. for 5 minutes. After shrimp alkaline phosphatase treatment, the genotyping reaction was combined with 0.76 μL of water, 0.2 μL of iPLEX termination mix (Sequenom, Inc., San Diego, Calif.), 0.04 μL of iPLEX Enzyme (Sequenom, Inc., San Diego, Calif.), 0.2 μL of 10× iPLEX Buffer, and 0.81 μL of 7-14 μM multiplexed extension primers. The MassEXTEND reaction was carried out at 94° C. for 2 minutes and then 99 cycles of 94° C. for 5 seconds, 52° C. for 5 seconds, and 72° C. for 5 seconds. The reaction mix was desalted by adding 3 mg of a cationic resin, SpectroCLEAN (Sequenom, Inc., San Diego, Calif.), and resuspended in 30 μL of water. Completed genotyping reactions were spotted in nanoliter volumes onto a matrix arrayed into 384 elements on a silicon chip (Sequenom SpectroCHIP), and the allele-specific mass of the extension products were determined by MALDI-TOF MS. Analysis of data was accomplished using the SPECTROTYPER software.

Haplotype analysis was performed using SAS/Genetics 9.1 (SAS, Cary, N.C.).

### Results

Fifty-two patients (56 eyes) met all entry criteria for this study. At the time of IVTA treatment, mean age was 70 years (standard deviation 13 years). Twenty-three (44%) patients were female. The patients were treated for a variety of retinal diseases, including choroidal neovascularization (frequently combined with photodynamic therapy) and macular edema due to diabetes mellitus, retinal vein occlusion, and other causes. Although uveitis patients were not specifically excluded from this study, no patient in this series had active uveitis at the time of treatment with IVTA.

Thirty eyes (54%) were phakic. Forty-seven eyes (84%) had no prior history of IVTA, and 9 eyes (16%) had been previously treated with IVTA, with an average interval of 1 year (standard deviation 0.8 years) between the previous IVTA treatment and entry into this study. Mean baseline IOP was 15 mmHg (standard deviation 3 mmHg). Mean maximum follow-up IOP was 22 mmHg (standard deviation 7 mmHg). Mean Δ IOP was 7 mmHg (standard deviation 6 mmHg). Δ IOP was less than 5 mmHg in 20 eyes (36%), 5-10 mmHg in 27 eyes (48%), and greater than 10 mmHg in 9 eyes (16%).

With respect to Δ IOP, the polymorphisms ER22/23EK, N363S, and at intron 3 were uninformative and were excluded from the statistical analysis. The polymorphisms N766N, Bc11, and at intron 4 passed the Hardy-Weinberg Equilibrium test, indicating good genotyping quality and normal population distribution of allelic frequency (Table 1).

The polymorphisms N766N and at intron 4, as well as the polymorphisms N766N and BclI, were found to be in strong linkage disequilibrium with each other, as could be expected based on their physical proximity (Table 2).

Discussion:

There is growing interest in the study of ophthalmic pharmacogenomics, particularly with respect to glaucoma medications. For example, in a study of 48 normal volunteers, the Arg389 homozygote genotype of the β1-adrenergic receptor correlated with a higher baseline IOP and a greater magnitude of response to treatment with betaxolol. In a study of 100 normal volunteers, the polymorphisms rs3753380 and rs3766355 of the prostaglandin Fₐ, receptor correlated with the magnitude of response to treatment with latanoprost. The results described herein are the first to seek a pharmacogenomic correlation with the steroid response following IVTA.

The primary outcome measure was A IOP, which was defined as the maximum IOP minus the baseline IOP. The maximum IOP was defined as the highest IOP recorded following IVTA for up to 1 year or until the eye was treated with any subsequent intravitreal injection, any intraocular surgery, or any therapy intended to lower IOP. The advantage of ΔTOP as a primary outcome measure is that it reduces the phenomenon of the steroid response to a single variable.

Patients under age 18, or patients using any medication to lower IOP, or with a history of glaucoma, suspected glaucoma, or ocular hypertension, were excluded from this study. Although uveitis patients were not specifically excluded, there were no patients with active uveitis in this series. Polymorphisms in the glucocorticoid receptor appeared to be reasonable candidates for a pilot study. Using dexamethasone suppression testing, ER22/23EK was associated with decreased glucocorticoid sensitivity, while N363S and BclI were associated with increased glucocorticoid sensitivity. In this study, statistically significant associations were not found between these 6 polymorphisms in the glucocorticoid receptor gene and Δ TOP following IVTA. The precise mechanism of steroid response remains elusive. There are many other possible candidate genes for study, such as other genes in the glucocorticoid pathway, including heat shock protein 90. See, also Table 3, for example. In addition, genome-wide screening on these samples will be performed to identify other candidate genes for study.

TABLE 1

| | Number | | | Minor | | Degrees | Probability > |
|---|---|---|---|---|---|---|---|
| | of | | | Allele | Chi- | of | Chi- |
| Locus | Alleles | PIC | Heterozygosity | Frequency | Square | Freedom | Square |
| N766N | 2 | 0.2241 | 0.2727 | 0.2571 | 0.2433 | 1 | 0.6218 |
| BclI | 2 | 0.3428 | 0.5303 | 0.4393 | 2.8338 | 1 | 0.0923 |
| Intron 4 | 2 | 0.3484 | 0.4394 | 0.4494 | 0.0326 | 1 | 0.8567 |

TABLE 2

| Locus 1 | Locus 2 | Haplotype | Frequency | Coefficient | D′ |
|---|---|---|---|---|---|
| Intron 4 | N766N | G-C | 0.1417 | 0.0901 | 0.9021 |
| Intron 4 | N766N | G-T | 0.1992 | −0.0901 | −0.9021 |
| Intron 4 | N766N | T-C | 0.0098 | −0.0901 | −0.9021 |
| Intron 4 | N766N | T-T | 0.6493 | 0.0901 | 0.9021 |
| Intron 4 | BclI | G-C | 0.2217 | −0.0082 | −0.0380 |
| Intron 4 | BclI | G-G | 0.1192 | 0.0082 | 0.0380 |
| Intron 4 | BclI | T-C | 0.4525 | 0.0082 | 0.0380 |
| Intron 4 | BclI | T-G | 0.2065 | −0.0082 | −0.0380 |
| N766N | BclI | C-C | 0.0099 | −0.0923 | −0.9034 |
| N766N | BclI | C-G | 0.1416 | 0.0923 | 0.9034 |
| N766N | BclI | T-C | 0.6644 | 0.0923 | 0.9034 |
| N766N | BclI | T-G | 0.1841 | −0.0923 | −0.9034 |

TABLE 3
A representative list of SNPs and their association with glaucoma

| | | | | P-value | | SNP |
|---|---|---|---|---|---|---|
| Chromosome | Transcript | Gene | Reps¹ | exponent | Gene | locati [symbol] |
| 10 | NM_020752 | GPR158 | | 10⁻⁸ | G protein-coupled receptor 158 | intron |
| | | | | | (member of the Glutamate clan) | |
| 12 | NM_001005494 | OR6C4 | | 10⁻⁴ | olfactory receptor, family 6, | ? |
| | | | | | subfamily C, member 4 | |
| 6 | NM_001039791 | FLJ45825 | 2 | 10⁻⁴ | FLJ45825 nonsense | ?? |
| | NM_153487 | MDGA1 | | | MDGA1 MAM domain | |
| | | | | | containing | |
| | | | | | glycosylphosphatidylinositol | |
| | | | | | anchor 1 | |
| 19 | NM_017721 | CC2D1A | | 10⁻⁴ | coiled-coil and C2 domain | intron |
| | | | | | containing 1A | |
| 12 | NM_005504 | BCAT1 | | 10⁻⁴ | branched chain aminotransferase | ? |
| | | | | | 1, cytosolic | |
| 2 | NM_001105 | ACVR1 | 2 | 10⁻⁴ | activin A receptor, type I | ?? |
| 3 | NM_006548 | IGF2BP2 | | 10⁻⁴ | insulin-like growth factor 2 | intron |
| | NM_001007225 | IGF2BP2 | | | mRNA binding protein 2 | |
| 17 | NM_025248 | SNIP | | 10⁻³ | SNAP25-interacting protein | intron |
| | NM_025152 | NUBPL | | 10⁻³ | NUBPL nucleotide binding | intron |
| | NM_080664 | C14orf126 | | | protein-like | |
| | | | | | C14orf126 chromosome 14 open | |
| | | | | | reading frame 126 | |
| 14 | NM_139179 | DAGLBETA | | 10⁻² | diacylglycerol lipase, beta | intron |
| 7 | NM_001960 | EEF1D | 2 | 10⁻² | eukaryotic translation elongation | intron |
| | NM_032378 | EEF1D | | | factor 1 delta (guanine | |
| | | | | | nucleotide exchange protein) | |
| 8 | NM_012175 | FBXO3 | 2 | 10⁻² | FBXO3 F-box protein 3 | ? |
| | NM_033406 | FBXO3 | | | LMO2 LIM domain only 2 | |
| | NM_005574 | LMO2 | | | (rhombotin-like 1) | |
| 11 | NM_178835 | LOC152485 | | 10⁻² | hypothetical protein LOC152485 | |
| | NM_005512 | LRRC32 | | 10⁻² | leucine rich repeat containing 32 | |
| | NM_003010 | MAP2K4 | | 10⁻² | mitogen-activated protein kinase | |
| | | | | | kinase 4 | |
| | NM_015550 | OSBPL3 | | 10⁻² | oxysterol binding protein-like 3 | |
| | NM_145320 | OSBPL3 | | | | |
| | NM_145321 | OSBPL3 | | | | |
| | NM_000918 | P4HB | | 10⁻² | procollagen-proline, 2- | |
| | | | | | oxoglutarate 4-dioxygenase | |
| | | | | | (proline 4-hydroxylase), beta | |
| | | | | | polypeptide | |
| | NM_004845 | PCYT1B | | 10⁻² | phosphate cytidylyltransferase 1, | |
| | | | | | choline, beta | |
| | NM_023078 | PYCRL | | 10⁻² | PYCRL pyrroline-5-carboxylate | |
| | NM_032862 | TIGD5 | | | reductase-like (PYCRL) | |
| | | | | | TIGD5 tigger transposable | |
| | | | | | element derived 5 | |
| | NM_031955 | SPATA16 | 2 | 10⁻² | spermatogenesis associated 16 | |
| | NM_005578 | LPP | | 10⁻² | LIM domain containing | |
| | | | | | preferred translocation partner in | |
| | | | | | lipoma | |
| | NM_004946 | DOCK2 | | 10⁻² | dedicator of cytokinesis 2 | |
| | NM_199051 | FAM5C | 2 | 10⁻² | family with sequence similarity | |
| | | | | | 5, member C | |
| | NM_001163 | APBA1 | | 10⁻² | amyloid beta (A4) precursor | |
| | | | | | protein-binding, family A, | |
| | | | | | member 1 | |
| | NM_152565 | ATP6V0D2 | | 10⁻² | ATPase, H+ transporting, | |
| | | | | | lysosomal 38 kDa, V0 subunit d2 | |
| | NM_020116 | FSTL5 | | 10⁻² | follistatin-like 5 (FSTL5), | |
| 5 | NM_001013717 | LOC441108 | | 10⁻² | LOC441108 hypothetical gene | ? |
| | NM_003060 | SLC22A5 | | | supported by AK128882 | |
| 5 | NM_003060 | SLC22A5 | 5 | 10⁻² | solute carrier family 22 (organic | Intron |
| | | | | | cation transporter), member 5 | 3′ UT [symbol] |
| | | | | | | Intron |
| | | | | | | Intron |
| | NM_014813 | LRIG2 | | 10⁻² | leucine-rich repeats and | |
| | | | | | immunoglobulin-like domains 2 | |
| | NM_013962 | NRG1 | | 10⁻² | neuregulin 1 | |
| | NM_002922 | RGS1 | | 10⁻² | RGS1 regulator of G-protein | |
| | NM_130782 | RGS18 | | | signalling 1 | |
| | | | | | RGS18 | |
| | NM_001009992 | ZNF648 | | 10⁻² | regulator of G-protein signalling | |
| | | | | | 18 | |
| | NM_002697 | POU2F1 | | 10⁻² | POU domain, class 2, | |
| | | | | | transcription factor 1 | |

¹Reps = number of repetitions of different SNPs in the same gene.
536 gene screening, 487 corticosteroids, 462 clinical (human) or epidemiologic studies: outcomes/complications [symbol]
indicates data missing or illegible when filed

### Example 2

### Pharmacogenomic Associations for Predicting the Steroid Response for Individual Patients

Purpose:

The phenomenon of increased intraocular pressure (TOP) following injection of intravitreal triamcinolone acetonide (IVTA) is an important clinical problem with a poorly understood etiology.

Methods:

A small DNA bank was created using peripheral blood samples from 53 patients treated with IVTA for a variety of retinal diseases. TOP was measured at baseline and at each subsequent visit for up to 1 year, or until the eye was treated with intraocular surgery, another intravitreal injection, or any medical therapy intended to lower TOP. ATOP was defined as the highest post-injection TOP minus the baseline TOP, with a positive value indicating a rise in TOP following IVTA. The peripheral blood samples were subjected to genome-wide DNA screening using the GENECHIP® Human Mapping 500K Array Set (Affymetrix, Santa Clara, Calif.).

Results:

Over 440,000 genes were screened. Forty-eight different single nucleotide polymorphisms (SNPs) within 33 genes were found to correlate (p<0.001) with magnitude of ΔIOP following IVTA. The strongest association involves a SNP within an as-yet poorly described G-protein coupled receptor (p=3.05×10⁻⁸). Four individual SNPs within a single transporter gene were identified (p between 5.59×10⁻⁴ and 2.81×10⁻⁵). Other genes with multiple SNPs included a translation elongation factor, an F-box protein, an oxysterol binding protein, and a solute carrier family gene. One group of 18 SNPs (FDR-corrected p=0.000709) divided the 53 samples into two groups, one containing the samples with the two highest ΔIOPs and one containing the other 51 samples.

Conclusions:

Several novel SNPs were identified which appear to correlate with magnitude of TOP response following IVTA to a highly statistically significant degree. Further studies are necessary to validate these associations and to identify the relevant genes. However, these data indicate a potential future ability to predict TOP elevation following IVTA for individual patients.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the will allow the reader to quickly ascertain the nature of the technical disclosure. It is submitted with the understanding that it will not be used to interpret or limit the scope or meaning of the following claims.

What is claimed is:

1. A composition of biomarkers comprising at least one marker:
NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6VOD2, ATPase, fr transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), a G-protein coupled receptor, a translation elongation factor, an F-box protein, an oxysterol binding protein, a solute carrier family gene, alleles, variants, mutants, homologs, fragments or complementary sequences thereof.

2. The biomarker composition of claim 1, wherein one or more markers comprise at least one polymorphic form.

3. The biomarker composition of claim 1, wherein at least one marker comprises at least one single nucleotide polymorphism.

4. A biomarker composition for predicting patient response to steroids comprising: NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6V0D2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), a G-protein coupled receptor, a translation elongation factor, an F-box protein, an oxysterol binding protein, a solute carrier family gene, alleles, variants, mutants, homologs, fragments or complementary sequences thereof.

5. The biomarker composition of claim 4, wherein one or more markers comprise at least one polymorphic form.

6. The biomarker composition of claim 4, wherein at least one marker comprises at least one single nucleotide polymorphism.

7. The biomarker composition of claim 4, further comprising glucocorticoid receptor single nucleotide polymorphisms and/or single nucleotide polymorphisms in BclI, N766N or intron 4.

8. A method of predicting risk for steroid response comprising:
obtaining a biological sample from a patient;
detecting a biomarker comprising at least one of: glucocorticoid receptor polymorphisms and/or single nucleotide polymorphisms in BclI, N766N, and/or intron 4 in the sample;
comparing the levels of the biomarkers in the sample to levels of the biomarker in a control sample; and,
predicting risk for steroid response.
The method of claim 4, wherein the biomarker further comprises detection of at least one of: NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6VOD2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), a G-protein coupled receptor, a translation elongation factor, an F-box protein, an oxysterol binding protein, a solute carrier family gene, alleles, variants, mutants, homologs, fragments or complementary sequences thereof.

9. The method of claim 8, wherein one or more markers comprise at least one polymorphic form.

10. The method of claim 8, wherein at least one marker comprises at least one single nucleotide polymorphism.

11. The method of claim 7, wherein steroid risk in a patient comprises a rise in intra-ocular pressure as compared to a normal individual.

12. The method of claim 7, wherein detection of one or more single nucleotide polymorphisms in at least one biomarker is predictive of risk in treating a patient with steroids.

13. A method of differentiating between a steroid responder versus a non-responder patient, comprising:
obtaining a biological sample from a patient;
detecting a biomarker comprising at least one of: glucocorticoid receptor polymorphisms and/or single nucleotide polymorphisms in BclI, N766N, and/or intron 4 in the sample;
comparing the levels of the biomarkers in the sample to levels of the biomarker in a control sample; and,
differentiating between a steroid responder versus a non-responder patient.
The method of claim 13, wherein the biomarker further comprises detection of at least one of: NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6VOD2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), a G-protein coupled receptor, a translation elongation factor, an F-box protein, an oxysterol binding protein, a solute carrier family gene, alleles, variants, mutants, homologs, fragments or complementary sequences thereof.

14. The method of claim 13, wherein one or more markers comprise at least one polymorphic form.

15. The method of claim 13, wherein at least one marker comprises at least one single nucleotide polymorphism.

16. The method of claim 13, wherein detection of one or more single nucleotide polymorphisms in at least one biomarker in a patient sample differentiates between a steroid responder versus a non-responder.

17. A method of identifying candidate therapeutic agents comprising:
providing a biological sample;
incubating the biological sample with a candidate therapeutic agent;
screening the biological sample for detection of one or more markers comprising: NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6V0D2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), a G-protein coupled receptor, a translation elongation factor, an F-box protein, an oxysterol binding protein, a solute carrier family gene, alleles, variants, mutants, homologs, fragments or complementary sequences thereof;
comparing the expression levels between the biological sample and control; and,
identifying a candidate therapeutic agent.

18. The method of claim 17, wherein one or more markers comprise at least one polymorphic form.

19. The method of claim 17, wherein at least one marker comprises at least one single nucleotide polymorphism.

20. The method of claim 17, wherein the biomarker further comprising at least one of: glucocorticoid receptor polymorphisms and/or single nucleotide polymorphisms in BclI, N766N, and/or intron 4 in the sample.

21. The method of claim 17, wherein the single nucleotide polymorphisms are detected using an immunoassay, hybridization, blotting, PCR, or biochip array.

22. The method of claim 17, wherein the biochip array is a nucleic acid array.
A kit for predicting risk for steroid responses comprising one or more of: NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6V0D2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); or, NM_{—}002697 (POU2F1, POU domain, class 2, transcription factor 1), a G-protein coupled receptor, a translation elongation factor, an F-box protein, an oxysterol binding protein, a solute carrier family gene, alleles, variants, mutants, homologs, fragments or complementary sequences thereof.

23. The kit of claim 22, wherein one or more markers comprise at least one polymorphic form.

24. The method of claim 22, wherein at least one marker comprises at least one single nucleotide polymorphism.

25. The kit of claim 22, wherein the biomarkers further comprise glucocorticoid receptor polymorphisms and single nucleotide polymorphisms in BclI, N766N and intron 4.

26. An antibody specific for markers or gene products thereof, wherein the markers comprise at least one single nucleotide polymorphism selected from the group consisting of:
NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6V0D2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4, variants, a G-protein coupled receptor, a translation elongation factor, an F-box protein, an oxysterol binding protein, a solute carrier family gene, alleles, variants, mutants, homologs, fragments or complementary sequences thereof.

27. A method of identifying, diagnosing or differentiating between steroid responders and non-responders comprising detecting at least one or more of:
NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6VOD2, ATPase, fr transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4, a G-protein coupled receptor, a translation elongation factor, an F-box protein, an oxysterol binding protein, a solute carrier family gene, alleles, variants, mutants, homologs, fragments or complementary sequences thereof.

28. The method of claim 27, wherein one or more markers comprise at least one polymorphic form.

29. The method of claim 27, wherein at least one marker comprises at least one single nucleotide polymorphism.

30. A biochip comprising any one or more of the nucleic acids, mutants, variants, fragments and corresponding peptides thereof comprising: NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6V0D2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4,), a G-protein coupled receptor, a translation elongation factor, an F-box protein, an oxysterol binding protein, a solute carrier family gene, alleles, variants, mutants, homologs, fragments or complementary sequences thereof.

31. A biochip comprising any one or more of antibodies and fragments thereof, or aptamers specific for gene products or nucleic acids of: NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6V0D2, ATPase, fr transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4,), a G-protein coupled receptor, a translation elongation factor, an F-box protein, an oxysterol binding protein, a solute carrier family gene, alleles, variants, mutants, homologs, fragments or complementary sequences thereof.

32. An agent modulating the expression or activity of the gene products by targeting one or more of the following molecules comprising:
NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6V0D2, ATPase, H⁺ transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4,), a G-protein coupled receptor, a translation elongation factor, an F-box protein, an oxysterol binding protein, a solute carrier family gene, alleles, variants, mutants, homologs, fragments or complementary sequences thereof.

33. The agent of claim 32, wherein one or more markers comprise at least one polymorphic form.

34. The agent of claim 32, wherein at least one marker comprises at least one single nucleotide polymorphism.

35. A method of identifying novel therapeutic compositions for use as steroids and/or modulating intraocular pressure comprising contacting a biochip comprising any one or more biomarkers: NM_{—}020752 (GPR158, G protein-coupled receptor 158); NM_{—}001005494 (OR6C4, olfactory receptor, family 6, subfamily C, member 4); NM_{—}001039791 (FLJ45825, FLJ45825 nonsense); NM_{—}153487 (MDGA1, MDGA1 MAM domain containing glycosylphosphatidylinositol anchor 1); NM_{—}017721 (CC2D1A, coiled-coil and C2 domain containing 1A); NM_{—}005504 (BCAT1, branched chain aminotransferase 1); NM_{—}001105 (ACVR1, activin A receptor, type I); NM_{—}006548 (IGF2BP2, insulin-like growth factor 2); NM_{—}001007225 (IGF2BP2, mRNA binding protein 2); NM_{—}025248 (SNIP, SNAP25-interacting protein); NM_{—}025152 (NUBPL, NUBPL nucleotide binding protein-like); NM_{—}080664 (C14orf126, C14orf126 chromosome 14 open reading frame 126); NM_{—}139179 (DAGLBETA, diacylglycerol lipase, beta); NM_{—}001960 (EEF1D, eukaryotic translation elongation factor 1 delta (guanine nucleotide exchange protein)); NM_{—}032378 (EEF1D, eukaryotic translation elongation factor 1 delta (guanine nucleotide exchange protein)); NM_{—}012175 (FBXO3, FBXO3 F-box protein 3); NM_{—}033406 (FBXO3); NM_{—}005574 (LMO2, LMO2 LIM domain only 2 (rhombotin-like 1)); NM_{—}178835 (LOC152485, hypothetical protein LOC152485); NM_{—}005512 (LRRC32, leucine rich repeat containing 32); NM_{—}003010 (MAP2K4, mitogen-activated protein kinase 4); NM_{—}015550 (OSBPL3, oxysterol binding protein-like 3); NM_{—}145320 (OSBPL3); NM_{—}145321 (OSBPL3); NM_{—}000918 (P4HB, procollagen-proline, 2-oxoglutarate 4-dioxygenase (proline 4-hydroxylase), beta polypeptide); NM_{—}004845 (PCYT1B, phosphate cytidylyltransferase 1, choline, beta); NM_{—}023078 (PYCRL, PYCRL pyrroline-5-carboxylate reductase-like); NM_{—}032862 (TIGD5, TIGD5 tigger transposable element derived 5); NM_{—}031955 (SPATA16, spermatogenesis associated 16); NM_{—}005578 (LPP, LIM domain containing preferred translocation partner in lipoma); NM_{—}004946 (DOCK2, dedicator of cytokinesis 2); NM_{—}199051 (FAM5C, family with sequence similarity 5, member C); NM_{—}001163 (APBA1, amyloid beta (A4) precursor protein-binding, family A, member 1); NM_{—}152565 (ATP6VOD2, ATPase, fr transporting, lysosomal 38 kDa, V₀ subunit d₂); NM_{—}020116 (FSTL5, follistatin-like 5); NM_{—}001013717 (LOC441108, LOC441108 hypothetical gene supported by AK128882); NM_{—}003060 (SLC22A5); NM_{—}003060 (SLC22A5, solute carrier family 22 (organic cation transporter), member 5); NM_{—}014813 (LRIG2, leucine-rich repeats and immunoglobulin-like domains 2); NM_{—}013962 (NRG1, neuregulin 1); NM_{—}002922 (RGS1, RGS1 regulator of G-protein signaling 1); NM_{—}130782 (RGS18); NM_{—}001009992 (ZNF648, regulator of G-protein signaling 18); NM_{—}002697 (POU2F1, POU domain, class 2, transcription factor 1), glucocorticoid receptor polymorphisms and/or single nucleotide polymorphisms in BclI, N766N and/or intron 4,), a G-protein coupled receptor, a translation elongation factor, an F-box protein, an oxysterol binding protein, a solute carrier family gene, alleles, variants, mutants, homologs, fragments or complementary sequences thereof.

36. The method of claim 35, wherein one or more markers comprise at least one polymorphic form.

37. The method of claim 35, wherein at least one marker comprises at least one single nucleotide polymorphism.

38. The method of claim 35, wherein the biochip comprises polynucleotides, aptamers, peptides or antibodies specific for any one or more of the biomarkers.
